(19) United States
(12) Patent Application Publication
(10) Pub. No.: US 2017/0071918 A1
(43) Pub. Date: Mar. 16, 2017

(54) Combination Use of CD47 Antibodies/Inhibitors with Sorafenib for Treatment of Liver Cancer

(71) Applicant: THE UNIVERSITY OF HONG KONG, Hong Kong (CN)

(72) Inventors: Oi Lin Irene Ng Lui, Hong Kong (CN); Terence Kin Wah Lee, Hong Kong (CN)

(21) Appl. No.: 14/856,137

(22) Filed: Sep. 16, 2015

(51) Int. Cl. A61K 31/44; A61K 31/167; A61K 39/395

(52) U.S. Cl. CPC A61K 31/44; A61K 39/39558; A61K 31/167

(57) ABSTRACT

The subject invention pertains to methods, and related compositions, for treating or inhibiting liver cancer, comprising administering an effective amount of sorafenib; and an inhibiting agent selected from a CD47 inhibiting agent, a NF-κB inhibiting agent, and combination thereof.

### BACKGROUND OF THE INVENTION

Liver cancer (hepatocellular carcinoma; HCC) is a major malignancy worldwide (1) and the front-line treatment for this disease is liver transplantation and surgical resection. However, most HCCs are inoperable, given that patients typically present at advanced stages, and even after surgical resection, the long-term prognosis of HCC remains unsatisfactory owing to its high recurrence rate. For advanced-staged HCC patients, they have only a 6-month median survival at diagnosis if HCC is left untreated (2). HCC treatment has recently entered a new era as a result of the development of the molecular targeting drug, sorafenib, which has demonstrated improvement in the survival of advanced HCC patients. To date, sorafenib is the only small-molecule inhibitor that has been approved by the U.S. Food and Drug Administration for HCC treatment. Two large-scale, phase III, randomized, clinical trials, including the SHARP trial (3) have demonstrated a survival benefit from treatment in advanced HCC patients (4). However, the survival benefit of the sorafenib treatment arm was modest (3, 4), partly owing to drug resistance (4, 5). Given that the exact mechanism for sorafenib resistance has not been reported, (6-8) studies on sorafenib-resistance mechanisms are urgently needed.

### BRIEF SUMMARY OF THE INVENTION

In one aspect, the present invention provides methods for treating or inhibiting liver cancer, comprising administering to a subject in need thereof an effective amount of: sorafenib; and an inhibiting agent selected from one or more CD47 inhibiting agent, one or more NF-κB inhibiting agent, or a combination thereof.

In another aspect, the present invention provides methods of preventing recurrence of or treating liver cancer in a subject, the methods comprising: administering to the subject an effective amount of sorafenib; and an inhibiting agent selected from one or more CD47 inhibiting agent, one or more NF-κB inhibiting agent, and combinations thereof, wherein the subject had surgical resection of a primary liver tumor, had undergone hepatectomy, and/or had liver transplantation.

In yet another aspect, the present invention provides methods for increasing sensitivity of liver cancer cells (which may include tumor initiating cells) to sorafenib treatment, comprising contacting the cells with an inhibiting agent selected from one or more CD47 inhibiting agent, one or more NF-κB inhibiting agent, or a combination thereof, either contemporaneously with, prior to, or after sorafenib treatment.

In some embodiments, the liver cancer (or liver cancer cells) is hepatocellular carcinoma (HCC). In some embodiments, the subject is a human.

In some embodiments the CD47 inhibiting agent is an anti-CD47 antibody, or antibody fragment thereof. In some embodiments, the antibody is either a chimeric, human, or humanized antibody. In some embodiments, the antibody is a monoclonal antibody. In some embodiments, the antibody fragment is selected from Fab, Fab′, Fab′-SH, F (ab′) 2, Fv, diabodies, single-chain antibody fragment, or a multispecific antibody comprising multiple different antibody fragments. In other embodiments, the anti-CD47 antibody is conjugated or covalently bound to a toxic moiety and/or a detectable moiety. An exemplary detectable moiety includes a radiolabel.

In some embodiments, the NF-κB inhibiting agent is IMD-0354.

In some embodiments, the CD47 and NF-κB inhibiting agents are selected from a respective antibody, a respective binding partner, a respective binding aptamer, an antisense nucleic acid molecule that inhibits the expression of the respective protein, a compound that inhibits the respective protein, and combinations thereof.

In a further aspect, the present invention provides pharmaceutical compositions comprising sorafenib; an inhibiting agent selected from a CD47 inhibiting agent, a NF-κB inhibiting agent, and combinations thereof; and a pharmaceutically acceptable carrier.

The methods and compositions herein described can be used in connection with pharmaceutical, medical, and veterinary applications, as well as fundamental scientific research and methodologies, as would be identifiable by a skilled person upon reading of the present disclosure. These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying figures.

FIG. 1 shows the establishment of sorafenib-resistant clones in vitro and in vivo. (A) Morphology of sorafenib-resistant Bel-7402 and Huh-7 cell lines established in vitro through continuous exposure to sorafenib (40×). (B) Successful establishment of sorafenib-resistant HCC cells (Bel-7402 and Huh-7) was confirmed by Annexin V staining, which showed that resistant cell lines were less sensitive to sorafenib. (C) XTT assay demonstrated a different effect of sorafenib on cell proliferation between mock-treated and sorafenib-resistant HCC cells. (D) By western blotting analysis, higher expression of Bax/Bcl2 ratio, cleaved caspase-9, and PARP in mock-treated HCC cells, was found when compared to sorafenib-resistant cells. (E) In vivo resistant clones were developed by SC, subcutaneous injection of tumor cells from PDTX (PDTX#1 and PDTX#5) in NOD-SCID mice that were orally fed 100 mg/kg of sorafenib daily for 31 days.

FIG. 2 shows that sorafenib-resistant HCC cells possess enhanced T-IC properties. (A) Sorafenib-resistant clones derived from Bel-7402 and Huh-7 cells showed increased ability to form tumors, when compared to their mock-treated counterparts. Mock-treated cells were injected into the left flank, whereas resistant cells were injected into the right flank. (B) Upon serial transplantation of 1×10³ cells derived from untreated or sorafenib-resistant residual tumors (PDTX#5) into other NOD/SCID mice, more tumors were found to result from sorafenib-resistant residual tumors (red arrows indicate tumor formation). Mock-treated cells were injected into the left flank, whereas resistant cells were injected into the right flank. (C) Spheroid formation after 8-10 days (40×) showed that spheroids formed by sorafenib-resistant clones were morphologically larger in size, compared to mock control. Greater numbers of spheres were also observed for sorafenib-resistant Bel-7402 (P=0.0003), Huh-7 (P=0.018), and PDTX#5 (P<0.0001) clones. (D) Light micrograph of cell invasion and migration assays, with staining of cells in the lower chamber (103). Fold differences in invasion and migration of sorafenib-resistant Bel-7402 (invasion, P=0.02; migration, P=0.01) and Huh-7 (invasion, P=0.005; migration, p=0.02) clones. (E) qPCR analysis revealed that both Bel-7402 and Huh-7 sorafenib-resistant clones had increased expression of common liver T-IC markers, such as CD24, CD133, and EpCAM, as well as the relatively more novel marker, CD47 (Bel-7402-CD47: P=0.01; CD133, P=0.01; EpCAM, P=0.002; Huh-7-CD47: P=0.04; Student t test). (F) Increased CD47 expression was found in sorafenib-resistant clones developed in vitro and in vivo, as analyzed by FCA. Analyzed cells were dissociated from the residual tumor, and 7-amino-actinomycin D was used to help exclude dead cells.

FIG. 3 shows sorafenib-resistant HCC cells exhibit NF-κB activation. (A) Luciferase reporter assays confirmed up-regulated NF-κB and CD47 promoter activity in Bel-7402 (NF-κB, P=0.003; CD47, P=0.005; Student t test). (B) Luciferase reporter assays confirmed up-regulated NF-κB and CD47 promoter activity in Huh-7 (NF-κB, P=0.015; CD47, P=0.018; Student t test). (C) Using western blotting analysis, phosphorylated p65 and IκBα were shown to be up-regulated in sorafenib-resistant clones, indicating increased NF-κB activation.

FIG. 4 shows NF-κB regulates CD47 expression. (A) TNF-α successfully activated NF-κB, as indicated by up-regulated phosphorylation of p65 and IκBα on western blotting. (B) CD47 detected by FCA further confirmed that CD47 expression was increased upon TNF-α treatment. (C) Luciferase reporter assay detected an increase in luciferase signal for NF-κB and CD47 upon addition of TNF-α for 1 hour (Bel-7402/NF-κB: P=0.002; CD47, P=0.004; PLC/PRF/5-NF-κB: P=0.003; CD47, P=0.018; t test). (D) IMD-0354 successfully suppressed NF-κB, as indicated by down-regulated phosphorylation of p65 and IκBα by western blotting. (E) CD47 detected by FCA further confirmed that CD47 expression was decreased upon IMD-0354 treatment. (F) Luciferase reporter assay detected a decrease in luciferase signal corresponding to NF-κB and CD47 expression upon addition of IMD-0354 (Huh-7/NF-κB: P=0.001; CD47, P=0.0005; MHCC97L/NF-κB: P=0.019; CD47, P=0.016, Student t test).

FIG. 5 shows direct interaction between NF-κB and CD47. (A) NF-κB bound to the CD47 promoter at putative binding sites 564 (site 1) and 1,740 bp (site 2) from the CD47 transcriptional start site in Bel-7402, Huh-7, and MHCC97L cells. (B) After using TNF-α for 1 hour to stimulate NF-κB, ChIP fold enrichment, relative to background, was found to be increased in the treatment group by qPCR (Bel-7402, site 1: 1.5- to 2.5-fold [P=0.004]; site 2: 1.6- to 2.5-fold [P=0.004]) (C) Conversely, addition of IMD-0354 to inhibit NF-κB decreased binding of NF-κB to the CD47 promoter in the treatment group (Huh-7, site 1: from 1.4- to 1.1-fold [P=0.024]; site 2: from 1.8- to 0.9-fold [P=0.006]; MHCC97L, site 1: from 4.3- to 1.4-fold [P=0.0006]; site 2: from 2.8- to 1.5-fold [P=0.03]). (D and E) Increased NF-κB site occupancy to the CD47 promoter in sorafenib-resistant HCC cells (Bel-7402, site 1: from 1.5- to 2.6-fold [P=0.008]; site 2: from 1.3- to 3.4-fold [P=0.039]; Huh-7, site 1: from 1.26- to 1.56-fold [P=0.024]; site 2: from 1.2- to 1.57-fold [P=0.013]).

FIG. 6 shows the effects of NF-κB and CD47 suppression on sorafenib sensitization. (A) Huh-7 and Bel-7402 cell lines showed increased sensitivity to sorafenib upon sorafenib and IMD-0354 cotreatment to suppress NF-κB. The effect of IMD-0354 alone was adjusted to a level that did not affect apoptosis on its own. (B) CD47 knockdown in MHCC97L and Huh-7 cells highly expressing CD47 was confirmed by FCA. (C) CD47 knock-down MHCC97L and Huh-7 clones showed increased sensitivity to sorafenib, as indicated by a greater percentage of Annexin V-positive cells. (D) In vitro response of sorafenib and anti-CD47 Ab (B6H12) combined treatment showed greater sensitivity to sorafenib, in comparison with anti-CD47 Ab and sorafenib treatment alone, as detected by apoptosis assays.

FIG. 7 shows anti-CD47 Ab in combination with sorafenib for HCC cancer therapy. (A) Cells from a PDTX (PDTX#5) were SC injected into NOD-SCID mice. Four treatment groups were employed, and IgG was used as the Ab control. Ab was given IP daily at 500 μg/mouse, whereas sorafenib was given orally at 100 mg/kg. SC tumor volume was measured and recorded every 2-3 days from initial treatment to tumor harvest (day 31). (B) Image of a harvested tumor; mice were sacrificed on day 31. (C) Serum was extracted from blood of mice at time of sacrifice (day 31) (D) Images (0.43 μm/pixel) of entire stained slide submitted to TUNEL assays, captured using an Aperio Scanscope CS imager.

FIG. 8 shows co-expression of CD47 with CD24, CD133 and EpCAM in sorafenib-resistant HCC cells. By FACS analysis of sorafenib-resistant Huh-7 cells, expression of CD47 overlaps with liver T-IC markers, CD133 (71.4%), CD24 (84.6%), EpCAM (43%); additionally, in sorafenib-resistant Bel-7402 cells, CD47+ cells also expressed the liver T-IC markers, CD133 (0.76%), CD24 (22.3%), EpCAM (44%).

FIG. 9 shows a schematic diagram showing the putative NF-κB binding sites (−564 bp referred to as site 1; −1740 referred to as site 2) on CD47 promoter.

FIG. 10 shows the feedback mechanism of NF-κB-induced CD47 in regulation of liver T-ICs. By western blot analysis, increased p-Erk (Thr202/Tyr204) and p-Akt (Ser473) were found in sorafenib-resistant Bel-7402 and Huh-7 cells, when compared with mock-treated counterparts. Upon knockdown of CD47, we found decrease in p-ERK (Thr202/Tyr204) and p-Akt (Ser473) in sorafenib-resistant Bel-7402 and Huh-7 cells, and their levels were comparable to that of mock-treated cells.

FIG. 11 shows the clinical correlation of NF-κB and CD47. NF-κB and CD47 expression of 52 clinical HCC tumor samples were analyzed with quantitative PCR. A positive correlation between NF-κB and CD47 was found using Pearson's correlation with statistical significance (R2=0.3659, p=0.0001)

FIG. 12 shows the effect of IMD-0354 on sorafenib sensitivity in mock-treated and sorafenib-resistant Bel-7402 and Huh-7 cells. (A) In Bel-7402, sorafenib-resistant cells were more resistant than mock-treated cells (14.9% vs 20.9%). After administration of IMD-0354 at 0.504, sensitization to sorafenib was observed in both mock-treated and sorafenib-resistant cells (20.9% to 30.8%; 14.9% to 32.9%). (B) In Huh-7, sorafenib-resistant cells were more resistant than mock-treated cells (28.4% vs 37.7%). After administration of IMD-0354 at 0.0504, sensitization to sorafenib was observed in both mock-treated and sorafenib-resistant cells (37.7% to 50.2%; 28.4% to 33.9%).

FIG. 13 shows the effect of CD47 knockdown on sorafenib sensitivity in mock-treated and sorafenib-resistant Bel-7402 and Huh-7 cells. (A) By flow cytometry analysis, CD47 expression was successfully repressed in mock-treated and sorafenib-resistant cells. (B) Upon administration of sorafenib at 15 μM in Bel-7402, sensitization to sorafenib was observed in both mock-treated and sorafenib-resistant cells after CD47 knockdown (25.2% to 38%; 19.1% to 37.6%). (C) In Huh-7, sensitization to sorafenib was observed in both mock-treated and sorafenib-resistant cells after CD47 knockdown (48.8% to 57.9%; 38.8% to 56.9%).

FIG. 14 shows the effect of anti-CD47 antibody on sorafenib sensitivity in mock-treated and sorafenib-resistant Bel-7402 and Huh-7 cells. (A) In Bel-7402, sorafenib-resistant cells were more resistant than mock-treated cells (13.2% vs 29.0%). After administration of anti-CD47 antibody at 2 μg/mL, sensitization to sorafenib was observed in both mock-treated and sorafenib-resistant cells (29.0% to 37.6%; 13.2% to 38.4%). In Huh-7, sorafenib-resistant cells were more resistant than mock-treated cells (35.1% vs 43.0%). After administration of anti-CD47 antibody at 2 μg/mL, sensitization to sorafenib was observed in both mock-treated and sorafenib-resistant cells (43.0% to 48.5%; 35.1% to 39.3%).

FIG. 15 shows that sorafenib increases CD47 expression in NF-κB dependent manner.

### BRIEF DESCRIPTION OF THE SEQUENCES

SEQ ID NO:1 is a nucleotide primer sequence useful in aspects of the present invention.

SEQ ID NO:2 is a nucleotide primer sequence useful in aspects of the present invention.

SEQ ID NO:3 is a nucleotide primer sequence useful in aspects of the present invention.

SEQ ID NO:4 is a nucleotide primer sequence useful in aspects of the present invention.

SEQ ID NO:5 is a nucleotide primer sequence useful in aspects of the present invention.

SEQ ID NO:6 is a nucleotide primer sequence useful in aspects of the present invention.

SEQ ID NO:7 is a nucleotide primer sequence useful in aspects of the present invention.

SEQ ID NO:8 is a nucleotide primer sequence useful in aspects of the present invention.

SEQ ID NO:9 is a nucleotide primer sequence useful in aspects of the present invention.

SEQ ID NO:10 is a nucleotide primer sequence useful in aspects of the present invention.

SEQ ID NO:11 is a nucleotide primer sequence useful in aspects of the present invention.

SEQ ID NO:12 is a nucleotide primer sequence useful in aspects of the present invention.

SEQ ID NO:13 is a nucleotide primer sequence useful in aspects of the present invention.

SEQ ID NO:14 is a nucleotide primer sequence useful in aspects of the present invention.

SEQ ID NO:15 is a nucleotide primer sequence useful in aspects of the present invention.

SEQ ID NO:16 is a nucleotide primer sequence useful in aspects of the present invention.

### DETAILED DISCLOSURE OF THE INVENTION

Aspects of the present invention are directed to compositions and methods for treatment of liver cancer, such as hepatocellular carcinoma (HCC).

Recently, solid evidence has emerged in support of a cancer stem cell/tumor-initiating cell (T-IC) model in leukemia and in a wide range of solid tumors, including HCC (9-12). T-ICs are believed to possess both cancer cell- and stem celllike characteristics, including tumor initiation, self-renewal, differentiation, and chemoresistance (13, 14). T-ICs have been implicated in recurrence and therapeutic resistance. Several recent studies support this hypothesis and have revealed that epithelial cell adhesion molecule (EpCAM)⁺ and label-retaining HCC cells are more resistant to sorafenib treatment (15, 16). Recently, CD47 has been identified as a novel therapeutic target for elimination of liver T-ICs (17). In addition to HCC, CD47 was reported to be a marker of T-ICs of leukemia and bladder cancer (18, 19). Recent reports have highlighted the therapeutic efficacy of targeting CD47 using anti-CD47 monoclonal antibody (mAb) in treatment of various cancer types (20). In addition, antagonizing CD47 rendered tumor cells susceptible to additional therapies and attack by the T-cell immune response (21).

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

In one aspect, the present invention provides methods for treating or inhibiting liver cancer, comprising administering to a subject in need thereof an effective amount of: sorafenib; and an inhibiting agent selected from one or more CD47 inhibiting agent, one or more NF-κB inhibiting agent, or a combination thereof.

The terms “effective amount” and “therapeutically effective amount,” used interchangeably, as applied to the inhibiting agents and pharmaceutical compositions described herein, mean the quantity necessary to render the desired therapeutic result. For example, an effective amount is a level effective to treat, cure, or alleviate the symptoms of a disorder for which the agent, or composition thereof, is being administered. Amounts effective for the particular therapeutic goal sought will depend upon a variety of factors including the disorder being treated and its severity and/or stage of development/progression; the bioavailability and activity of the specific agents or pharmaceutical composition used; the route or method of administration and introduction site on the subject; the rate of clearance of the specific agent or composition and other pharmacokinetic properties; the duration of treatment; inoculation regimen; drugs used in combination or coincident with the specific agent or composition; the age, body weight, sex, diet, physiology and general health of the subject being treated; and like factors well known to one of skill in the relevant scientific art. Some variation in dosage will necessarily occur depending upon the condition of the subject being treated, and the physician or other individual administering treatment will, in any event, determine the appropriate dosage for an individual patient.

As used herein, the term “subject” refers to an animal. Typically, the terms “subject” and “patient” may be used interchangeably herein in reference to a subject. As such, a “subject” includes a human that is being treated for a disease as a patient. The term “animal,” includes, but is not limited to, mouse, rat, dog, cat, rabbit, pig, monkey, chimpanzee, and human.

As used herein, “treatment” or “treating” refers to arresting or inhibiting, or attempting to arrest or inhibit, the development or progression of a disease and/or causing, or attempting to cause, the reduction, suppression, regression, or remission of a disease and/or a symptom thereof. As would be understood by those skilled in the art, various clinical and scientific methodologies and assays may be used to assess the development or progression of a disease, and similarly, various clinical and scientific methodologies and assays may be used to assess the reduction, regression, or remission of a disease or its symptoms. “Treatment” refers to both therapeutic treatment and prophylactic or preventative measures. Those in need of treatment include those already with the disease as well as those prone to have the disease or those in whom the disease is to be prevented. In at least one embodiment, the disease being treated is cancer. In other embodiments, the disease being treated is HCC.

In another aspect, the present invention provides methods of preventing recurrence of or treating liver cancer in a subject, the methods comprising: administering to the subject an effective amount of sorafenib; and an inhibiting agent selected from one or more CD47 inhibiting agent, one or more NF-κB inhibiting agent, and combinations thereof, wherein the subject had surgical resection of a primary liver tumor, had undergone hepatectomy, and/or had liver transplantation.

In yet another aspect, the present invention provides methods for increasing sensitivity of liver cancer cells (which may include tumor initiating cells) to sorafenib treatment, comprising contacting the cells with an inhibiting agent selected from one or more CD47 inhibiting agent, one or more NF-κB inhibiting agent, and combinations thereof, either contemporaneously with, prior to, or after sorafenib treatment.

In some embodiments, the liver cancer (or liver cancer cells) is hepatocellular carcinoma (HCC). In some embodiments, the subject is a human.

Administration (or contacting) may be locally (confined to a single cell or tissue) and/or systemically in the subject. It may be desirable to administer the inhibiting agents (e.g., antibodies) and pharmaceutical compounds of the invention locally to the area in need of treatment, such as areas including one or more tumor. This method of administration may be achieved by, for example, and not by way of limitation, local infusion during surgery, topical application (e.g., in conjunction with a wound dressing after surgery), by injection, by catheter, or by means of an implant (e.g., a porous membrane). When administering inhibiting agents, care must be taken to use materials which do not absorb the inhibiting agents, thus allowing for effective release, or delayed release if so desired by the materials used.

In some embodiments, the inhibiting agents or pharmaceutical composition can be delivered in a controlled release system. Such methods may include the use of a pump for administration (e.g., use of an intravenous drip). In another embodiment, a controlled release system can be placed in the proximity of the therapeutic target (e.g., a tumor), requiring only a fraction of the dose required if dosed systemically.

The inhibiting agents and compositions of the invention can be used to treat, alleviate, inhibit or prevent cell-proliferative disorders, such as cancer. The cell-proliferative disorder may be liver cancer, such as hepatocellular carcinoma. Furthermore, it would be understood by those skilled in the art that the therapeutic methods described would not only apply to treatment in a subject, but could be applied to cell cultures, organs, tissues, or individual cells in vivo, ex vivo, or in vitro.

The amount of the inhibiting agents or pharmaceutical compound of the invention which will be effective in the treatment, inhibition and/or prevention of a disease or disorder can be determined by standard clinical techniques. Additionally, in vitro assays may be employed to help identify optimal dosage ranges. The precise dose to be utilized will also depend on the route of administration, and the seriousness of the disorder, and should also be decided according to the sound medical judgment of the clinician and each patient's individual circumstances. The specific therapeutically effective dose level for any particular patient will depend upon a variety of factors including: the type and degree of the response to be achieved; the specific composition and other agent(s), if any, employed; the age, weight, general health, sex and diet of the patient; the time of administration, route of administration, and rate of excretion of the composition; the duration of the treatment; other drugs (such as a chemotherapeutic agents) used in combination or coincidental with the composition; and any other factors well known in the medical arts. Effective dosages may also be extrapolated from dose-response curves derived from in vitro or animal model testing systems.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (VL) and variable heavy chain (VH) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. Embodiments of the present invention include monoclonal antibodies. Humanized, chimeric, human, or otherwise-human-suitable antibodies are also embodied by the present invention. “Antibodies” also includes any fragment or derivative of any of the herein described antibodies.

A human-suitable antibody refers to any antibody, derivatized antibody, or antibody fragment that can be safely used in humans for, e.g. the therapeutic methods described herein. Human-suitable antibodies include all types of humanized, chimeric, or fully human antibodies, or any antibodies in which at least a portion of the antibodies is derived from humans or otherwise modified so as to avoid the immune response that is generally provoked when native non-human antibodies are used.

For the purposes of the present invention, a “humanized” or “human” antibody refers to an antibody in which the constant and variable framework region of one or more human immunoglobulins is fused with the binding region, e.g. the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In some embodiments the CD47 inhibiting agent is an anti-CD47 antibody, or antibody fragment thereof. In some embodiments, the antibody is either a chimeric, human, or humanized antibody. In some embodiments, the antibody is a monoclonal antibody. In some embodiments, the antibody fragment is selected from Fab, Fab′, Fab′-SH, F (ab′) 2, Fv, diabodies, single-chain antibody fragment, or a multispecific antibody comprising multiple different antibody fragments.

In other embodiments, the anti-CD47 antibody is conjugated or covalently bound to a toxic moiety and/or a detectable moiety, such as a fluorescent moiety, a radioisotope or an imaging agent; or to a solid support, such as agarose beads or the like. Methods for conjugation or covalent bonding of these other agents to antibodies are well known in the art. Conjugation to a toxin is useful for targeted killing of cancer cells. Conjugation to a detectable moiety is useful, inter alia, when an antibody of the invention is used for diagnostic purposes. Such purposes include, but are not limited to, assaying biological samples, e.g., a tissue biopsy or serum, for the presence of CD47, and detecting the presence or level of CD47 in an individual. Such methods are useful, e.g., for diagnosing conditions caused by or associated with an increase in CD47 expression or levels, e.g. cancer.

In some embodiments, the NF-κB inhibiting agent is IMD-0354.

In some embodiments, the CD47 and NF-κB inhibiting agents are selected from a respective antibody, a respective binding partner, a respective binding aptamer, an antisense nucleic acid molecule that inhibits the expression of the respective protein, a compound that inhibits the respective protein, and combinations thereof. Inhibitors of CD47: The anti-CD47 antibody (B6H12, Hu5F9-G4); Anti-CD47 VIVO morpholino (GeneTools); Anti-SIRPa antibodies. Inhibitors of NFκB activation: BAY 11-7082; BAY 11-7085; Helenalin; JSH-23; QNZ; IMD-0354; Celastrol; Triptolide; MG132; Pyrrolidinedithiocarbamate ammonium; Piceatannol; Cardamonin; Withaferin A.

In a further aspect, the present invention provides pharmaceutical compositions comprising sorafenib; an inhibiting agent selected from a CD47 inhibiting agent, a NF-κB inhibiting agent, and combinations thereof; and a pharmaceutically acceptable carrier. Such compositions may be useful for both diagnostic and therapeutic purposes.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

Pharmaceutically acceptable carriers that may be used in these compositions include, but are not limited to, sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, sucrose, gelatin, lactose, malt, rice, flour, chalk, silica gel, sodium stearate, glycerol monostearate, talc, sodium chloride, glycerol, propylene, glycol, water, ethanol and the like. The pharmaceutical composition may also contain wetting or emulsifying agents or suspending/diluting agents, or pH buffering agents, or agents for modifying or maintaining the rate of release of the antibody. These compositions can take the form of solutions, suspensions, emulsion, tablets, pills, capsules, powders, sustained-release formulations and the like. Formulations can include standard carriers such as pharmaceutical grades of mannitol, lactose, sodium saccharine, starch, magnesium stearate, cellulose, magnesium carbonate, etc. Such compositions will contain a therapeutically effective amount of the antibody together with a suitable amount of carrier so as to provide the proper form to the patient based on the mode of administration to be used.

If for intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachette indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

Moreover, if a packaging material is utilized to package the pharmaceutical composition, it may be biologically inert or lack bioactivity, such as plastic polymers, silicone, etc. and may be processed internally by the subject without affecting the effectiveness of the antibody packaged and/or delivered therewith.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The present invention shows that targeting liver T-ICs with an anti-CD47 antibody (Ab) is a way to evade sorafenib resistance in cancers. The present invention examines whether the T-IC population was enriched in sorafenib-resistant clones that were established both in vitro and in vivo through continuous administration of sorafenib. These clones were endowed with increased T-IC properties and consistent up-regulation of CD47 expression, which was regulated by nuclear factor kappa B (NF-κB) through its direct binding to a specific response element in the CD47 promoter. Consistently, CD47 levels were significantly correlated with NF-κB levels in a cohort of HCC clinical samples. Suppression of CD47 expression by a lentivirus-based knockdown approach in MHCC97L and Huh-7 cells showed increased sensitivity to sorafenib. Similarly, HCC cells were sensitized to sorafenib treatment upon administration of an NF-κB inhibitor (IMD-0354). Furthermore, in vitro ligation of CD47 with an anti-CD47 Ab (B6H12) sensitized HCC cells to the effect of sorafenib. Using a patient-derived tumor xenograft (PDTX) model, anti-CD47 Ab (B6H12) inhibited the growth of HCC cells and exerted a synergistic effect when combined with sorafenib treatment. This invention shows that NF-κB-mediated CD47 up-regulation promotes sorafenib resistance in HCC and that its blockade enhances the effect of sorafenib.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

Cell Lines and Cell Culture.

The human HCC cell lines MHCC97L (Liver Cancer Institute, Fudan University, China), Huh-7 (Japan Cancer Research Bank, Tokyo, Japan), PLC/PRF/5 (Japanese Cancer Research Bank), and Bel-7402 (Shanghai Institute of Cell Biology, Chinese Academy of Sciences) were maintained in DMEM containing high glucose (Gibco BRL, Grand Island, N.Y.), 10% heat-inactivated fetal bovine serum (Gibco BRL), 100 mg/mL penicillin G, and 50 mg/mL streptomycin (Gibco BRL) at 37° C. in a humidified atmosphere containing 5% CO₂.

Plasmids and Reagents.

Sorafenib was obtained from LC Laboratories (Woburn, Mass., USA). CD47 luciferase promoter was purchased from GeneCopoeia (Rockville, Md., USA). Reporter plasmids pNF-κB-Luc and pRL-cytomegalovirus were from Stratagene (LaJolla, CA, USA) and Promega (Madison, Wis., USA) respectively, TNF-α and IMD-0354 were purchased from R&D systems (Minneapolis, Minn., USA) and Sigma (Saint Louis, Mo., USA), respectively. Monoclonal antibody against CD47 (B6H12) and IgG control (MOPC-21) was purchased from BioXcell (West Lebanon, N.H., USA).

Primer Sequences.

| Genes | Forward primer (5′-3′) | Reverse primer (5′-3′) |
|---|---|---|
| β-actin | GTGGGGCGCCCCAGGCACCA SEQ ID NO: 1 | CTCCTTAATGTCACGCACGATTTC SEQ |
| | | ID NO: 2 |
| CD24 | TGAAGAACATGTGAGAGGTTTGAC SEQ ID | GAAAACTGAATCTCCATTCCACAA |
| | NO: 3 | SEQ ID NO: 4 |
| CD47 | CAATCACGTAAGGGTCTCATAGG | GATGGACTCCGATTTGGAGA |
| | SEQ ID NO: 5 | SEQ ID NO: 6 |
| CD133 | TGGATGCAGAACTTGACAACGT | ATACCTGCTACGACAGTCGTGGT |
| | SEQ ID NO: 7 | SEQ ID NO: 8 |
| EpCAM | TGATCCTGACTGCGATGAGAG SEQ ID NO: 9 | CTTGTCTGTTCTTCTGACCCC |
| | | SEQ ID NO: 10 |
| NFκB p65 | AGCACCATCAACTATGAT SEQ ID NO: 11 | GAGTTATAGCCTCAGGGT |
| | | SEQ ID NO: 12 |
| CD47 promoter binding site 1 | GGACACCTAGGCTTTCACCA SEQ ID NO: 13 | AGGGAAGAGCAGAGCGAGTA |
| | | SEQ ID NO: 14 |
| CD47 promoter binding site 2 | AGAGCAGCTGGTTGGCTAAA SEQ ID NO: 15 | GAACATCACCCGATCTCCAC |
| | | SEQ ID NO: 16 |

Patient Samples.

Human HCC samples were collected at the time of surgical resection at Queen Mary Hospital, the University of Hong Kong, from 1992 to 2001. After collection from surgical resection, all samples were immediately snap-frozen in liquid nitrogen before storage at −80° C., with Institutional Review Board (IRB) approval.

In Vivo Therapeutic Targeting and its Combined Effect with Sorafenib.

The xenograft was established in 4- to 6-week-old nonobese diabetic severe combined immonudeficiency (NOD/SCID) mice using the PDTX#5. Treatment was started once the size of the xenograft reached approximately 5×5 mm (length×width). Mice were then randomly assigned into four groups, each consisting of 5 mice. To evaluate the effect of anti-CD47 Ab alone, two groups received the following daily administrations by intraperitoneal (IP) injection: (1) 500 μg/mouse control immunoglobulin G (IgG; MOPC-21; group A) and (2) 500 μg/mouse anti-CD47 Ab (B6H12; BioXCell, Beverly, Mass.; group B). To evaluate the combined effect of anti-CD47 Ab with sorafenib, an additional two groups were included that received oral administration of (3) 100 mg/kg of sorafenib plus 500 μg/mouse control IgG (group C) and (4) 100 mg/kg of sorafenib plus 500 μg/mouse anti-CD47 Ab (group D). Tumor growth was measured and recorded over 31 days of various treatments and the treatment effects were observed.

Quantitative PCR (qPCR) Analysis.

Total RNA was isolated using TRIzol reagent according to the manufacturer's protocol (Invitrogen, Carlsbad, Calif.). Complementary DNA (cDNA) was synthesized using a GeneAmp® Gold RNA PCR Kit (Applied Biosystems, Foster City, Calif.) according to the manufacturer's instructions and then subjected to qPCR with a SYBR Green PCR kit with primers specific to the sequences of which are provided in Supplementary Materials. The amplification protocol consisted of incubations at 94° C. for 15 seconds, 63° C. for 30 seconds, and 72° C. for 60 seconds. Incorporation of the SYBR Green dye into PCR products was monitored in real time with an ABI 7900HT Sequence Detection System and SDS 1.9.1 software (Applied Biosystems) and subsequently analyzed using RQ Manager 1.2 software (Applied Biosystems), thereby allowing the threshold cycle (CI′) at which exponential amplification of the products began to be determined. The amount of target cDNA was calculated relative to that of β-actin cDNA.

ELISA.

Serum samples from different groups of mice were evaluated. Serum samples were kept at −80° C. prior to use. AFP in serum was measured using a commercial ELISA kit, purchased from Aviva Systems Biology (San Diego, Calif.). Sample, reagent, and buffer preparation were performed according to manufacturer manuals. Microwell plates were read at 450 nm wavelength with an Infinite F200 Tecan multiplate reader (Tecan Austria GmbH, Salzburg, Austria). Protein concentrations were determined from a standard curve generated from standards supplied with the kit.

Quantification of Apoptosis.

The TUNEL assay was performed to detect apoptotic cells using in situ cell death detection kit (Roche Diagnostics, Indianapolis, Ind.). Briefly, paraffin-embedded tissues were fixed with 15 μg/mL proteinase K in 10 mM Tris/HCl at pH 7.4. The slides were then incubated with the TUNEL reaction mixture for 1 h at 37° C. After washing, the slides were incubated with a horseradish peroxidase-conjugated anti-fluorescein antibody for 30 min at 37° C. Stained slides were imaged on an Aperio Scanscope® CS imager (Vista, Calif.), generating 0.43 μm/pixel whole slide images. These images were compiled, and number of apoptotic nuclei was quantitated using the Aperio Spectrum® software with a pixel count algorithm.

Chromatin Immunoprecipitation (ChIP) Assay.

The cells were processed for ChIP assays according to Upstate CHIP protocol. Briefly, cells were cross-linked for 10 min with 1% formaldehyde and lysed. Lysate pellets were resuspended and sonicated with a Microson sonifier XL-2000 (Misonix) for 10 cycles (ON/OFF 30 seconds; amplitude 10 microns). Protein-DNA complexes were immunoprecipitated using p65 (C-20) antibody (Santa Cruz Technology, Santa Cruz, Calif.) or Normal rabbit IgG (Thermo Fisher Scientific, Rockford, Ill.) bound to protein A agarose, eluted, and digested with proteinase K. For qPCR analysis of the ChIP DNA samples before amplicon generation, QIAquick-purified immunoprecipitates were dissolved in 30 μl of water. Standard PCR reactions using 24 of the immunoprecipitated DNA were performed with a SYBR Green PCR kit (Applied Biosystems) using primers which generates a PCR amplicon flanking −564 and -1740 base pairs upstream of the NM 001777 (CD47) transcription start site based on bioinformatics analysis on the Human Genome Build from the UCSC Genome Browser February 2009 (GRCh37/hg19) Assembly. Calculation of NF-κB occupancy on the CD47 promoter was performed according to the ChIP-qPCR primer assay data analysis template from Superarray Bioscience (Frederick, Md.).

Nuclear and Cytosolic Fractionation.

Cytosolic protein fraction was first extracted from cells

upon lysis with Buffer A (0.5 mM DTT, 10 mM HEPES, 10 mM KCl 1.5 mM MgCl2, pH7.9) for 15 minutes on ice and 0.5% NP-40 was added before extraction by centrifugation. The remaining pellet was treated with RIPA buffer (50 mM Tris-HCl pH7.4, 1% NP-40, 0.05% SDS 150 mM NaCl, 1 mM EDTA) for 30 minutes on ice and the nuclear fraction was obtained by centrifugation.

XTT Assay.

Mock-treated and sorafenib-resistant HCC cells were seeded on 96-well plates and appropriate concentrations of sorafenib ranging from 5 to 25 μM were then added. After 48 h, (2,3-Bis-(2-Methoxy-4-Nitro-5-Sulfophenyl)-2H-Tetrazolium-5-Carboxanilide) (XTT) dye, at a concentration of 5 mg/mL (Sigma-Aldrich, St Louis, Mo.), was added and the plates were incubated for 12 h in a moist chamber at 37° C. Optical density was determined by eluting the dye with dimethyl sulfoxide (Sigma-Aldrich) and the absorbance was measured at 570 nm. At least three independent experiments were performed.

Western Blot Analysis.

Western blots were developed using an ECL Plus kit (Amersham Biosciences, Piscataway, N.J.). The primary antibodies included mouse anti-human p65 (Ser536), mouse anti-human p-Akt (Ser473), anti-human p-Erk (Thr202/Tyr204), rabbit anti-human Bcl-2, rabbit anti-human cleaved caspase-9, rabbit anti-human cleaved PARP, mouse anti-human p-IκBα (Ser32/36) (Cell Signal Technology) and mouse anti-human α-tubulin, rabbit anti-human Bax, rabbit anti-human SP1 (PEP2) (Santa Cruz Technology, Santa Cruz, Calif.). After washing, the membrane was incubated with horseradish peroxidase-conjugated anti-mouse or rabbit antibody (Amersham) and then visualized with enhanced chemiluminescence plus according to the manufacturer's protocol.

Luciferase Reporter Assay.

NF-κB promoter plasmid pNF-κB-Luc or CD47 luciferase promoter plasmid were transfected together with pRL-cytomegalovirus as normalization using Lipofectamine 2000 (Thermo Fisher Scientific). Cell were incubated for 48 hours prior to detection by Dual-Luciferase Reporter Assay System Kit (Promega) according to manufacterer's protocol. Gaussia Luciferase Assay Kit (GeneCopoeia) was used for CD47 detection. Samples were detected with Infinite F200 Tecan Multiplate reader (Tecan Austria GmbH).

Sphere Formation Assay.

A total of 200 single HCC cells were plated onto 24-well polyHEMA (Sigma)-coated plates. Cells were grown in DMEM/F12 medium (Invitrogen, Carlsbad, Calif.) for 10 days supplemented with 4 μg/mL insulin (Sigma-Aldrich, St. Louis, Mo.), B27 (Invitrogen, Carlsbad, Calif.), 20 ng/mL EGF (Sigma-Aldrich, St. Louis, Mo.), and 20 ng/mL basic FGF (Invitrogen, Carlsbad, Calif.).

Migration and Invasion Assays.

The migration assay was performed as described. The cell invasion assay was performed with self-coated Matrigel (BD Biosciences, San Jose, Calif.) on the upper surface of a transwell chamber. The invasive cells that had invaded through the extracellular matrix layer to the lower surface of the membrane were fixed with 2% PFA in PBS and stained with crystal violet. Photographs of four randomly selected fields of the fixed cells were captured and cells were counted. The experiments were repeated independently three times.

Tumorigenicity Assay.

In vivo evaluation of tumorigenicity was done with NOD-SCID mice by induction of tumor xenograft. Cells were suspended in 1:1 culture medium and BD Matrigel Matrix (BD Biosciences) and subcutaneously injected into the flanks of the NOD-SCID mice which were kept under observation. Tumors were harvested at the end of experiment for documentation.

Flow Cytometric Analysis.

Phycoerythrin (PE)-conjugated CD47 (BD PharMingen, San Jose, Calif.), APC-conjugated CD133 (MACS Miltenyi Biotec), FITC-conjugated CD24 (MACS Miltenyi Biotec), FITC-conjugated EPCAM (Becton Dickinson), were incubated in phosphate-buffered saline (PBS) containing 2% fetal bovine serum (FBS) for 30 minutes at 4° C. Isotype-matched mouse immunoglobulins served as controls. The samples were analyzed using a Canto II flow cytometer and FACSdiva software (BD Biosciences, San Jose, Calif.).

Lentiviral-Based Transfection into HCC Cells.

For suppression of CD47 in HCC cells, lentiviral particles (DFCI-Broad RNAi Consortium, Boston) expressing shRNAs against human CD47 were used to downregulate CD47 mRNA. Transduced cells were selected with 2 μg/mL puromycin.

Annexin V Staining.

Cells were stained in binding buffer, proipidium iodide (PI) and FITC-conjugated Annexin V as provided by the Annexin-V-FLUO+S Staining Kit (Roche Diagnostics) according to manufacturer's instructions. Analysis was determined using a Canto II flow cytometer and FACSdiva software (BD Biosciences).

Establishment of Sorafenib-Resistant Clones.

Sorafenib-resistant clones were established both in vitro and in vivo through continuous treatment of cells with sorafenib. In vitro establishment was accomplished through gradual increase of sorafenib concentration over four months with DMSO as a mock-treatment counterpart until maximum tolerated dosage was reached. In vivo establishment was accomplished by subcutaneous injection of PDTX sample into NOD-SCID mice followed by treatment with sorafenib for a month once small tumors of 5×5 mm were formed. Residual tumor from the sorafenib treated group were considered relatively more resistant than the non-treated group.

Tumor Processing for Isolation of Single Cells.

Tumors harvested from in vivo experiments were processed for single cell isolation. Tumors were first cut into small pieces and transferred to gentleMACS C tube (Miltenyi Biotec GmbH) with 4 μg/ml Liberase and 20 μg/ml DNaseI (Roche) and dissociated with gentleMACS dissociator (Miltenyi) according to instructions provided by the manufacturer to obtain single cells

Statistical Analysis.

Statistical significance of the results obtained for the dual-luciferase reporter, chromatin immunoprecipitation (ChIP) promoter-binding, sphere formation, invasion and migration assays was determined by Student t test using Microsoft Office Excel software (Microsoft Corporation, Redmond, Wash.). Results are shown as means and standard deviations, and P values less than 0.05 were considered statistically significant. Pearson's correlation analysis was used to determine the correlation among NF-κB, p65, and CD47 expression using Prism 6 software (v.6.04; GraphPad Software Inc., La Jolla, Calif.).

### Example 1

### In Vitro and In Vivo Establishment of Sorafenib-Resistant HCC Cells

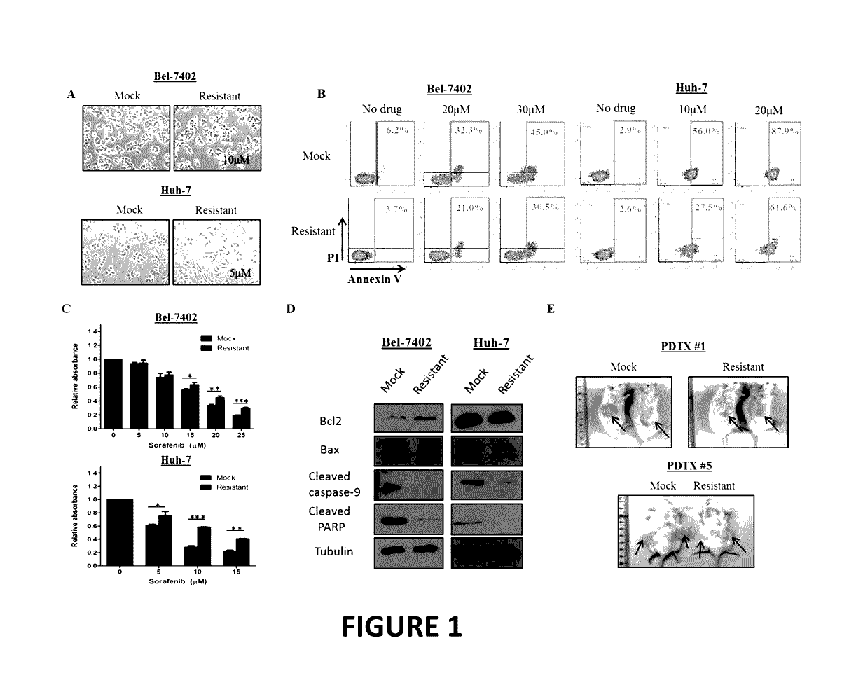

To determine whether liver T-ICs are enriched upon sorafenib treatment, we developed sorafenib-resistant HCC cells both in vitro and in vivo. In vitro establishment of sorafenib-resistant clones was accomplished in Huh-7 and Bel-7402 cells by continuous administration of gradually increasing sorafenib concentration over 4 months. Bel-7402 and Huh-7 cells were constantly incubated up to 10 and 5 μM of sorafenib, respectively. Upon incubation with sorafenib for 6 months, we established sorafenib-resistant cells derived from Bel-7402 and Huh-7 cells. Using phase-contrast microscopy, sorafenib-resistant cells exhibited an elongated spindle shape, in contrast to the mock-control cells, which had a rounded appearance (FIG. 1(A)). Using Annexin V staining, sorafenib-resistant cells were less sensitive to sorafenib at the two different doses, when compared to mock-control cells, indicating successful establishment of sorafenib-resistant HCC cells (FIG. 1(B)). To further confirm successful establishment of sorafenib-resistant cells, the effect of sorafenib on cell proliferation was evaluated. By XTT assay, we found that the growth-suppressive effect of sorafenib was significantly greater in mock-treated cells (FIG. 1(C)). In addition, the effect of sorafenib on apoptosis in mock-treated and sorafenib-resistant cells was evidenced by differential expression of apoptosis cascade proteins, including B-cell lymphoma 2 (Bcl2), Bcl2-associated X protein (Bax), cleaved caspase-9, and poly(ADP-ribose) polymerase (PARP; FIG. 1(D)). For in vivo selection, a sorafenib-resistant xenograft model was established using patient-derived HCC tumor xenografts (PDTX#1 and PDTX#5) using a high dose of sorafenib. When tumors reached 5 mm in diameter, mice were orally administered sorafenib at a dose of 100 mg/kg. After oral administration of sorafenib for 31 days, we found that treatment resulted in tumor inhibition among the xenografts (FIG. 1(E)). Using this in vivo treatment, we enriched the proportion of sorafenib-resistant HCC cells in residual tumors.

### Example 2

### CD47 is Up-Regulated in Sorafenib-Resistant HCC Cells Endowed with Enhanced T-IC Properties

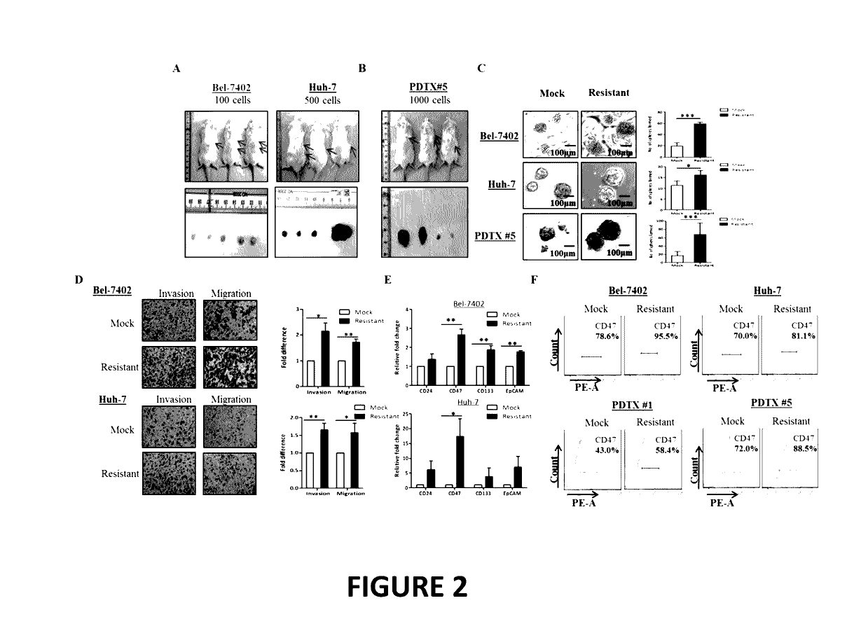

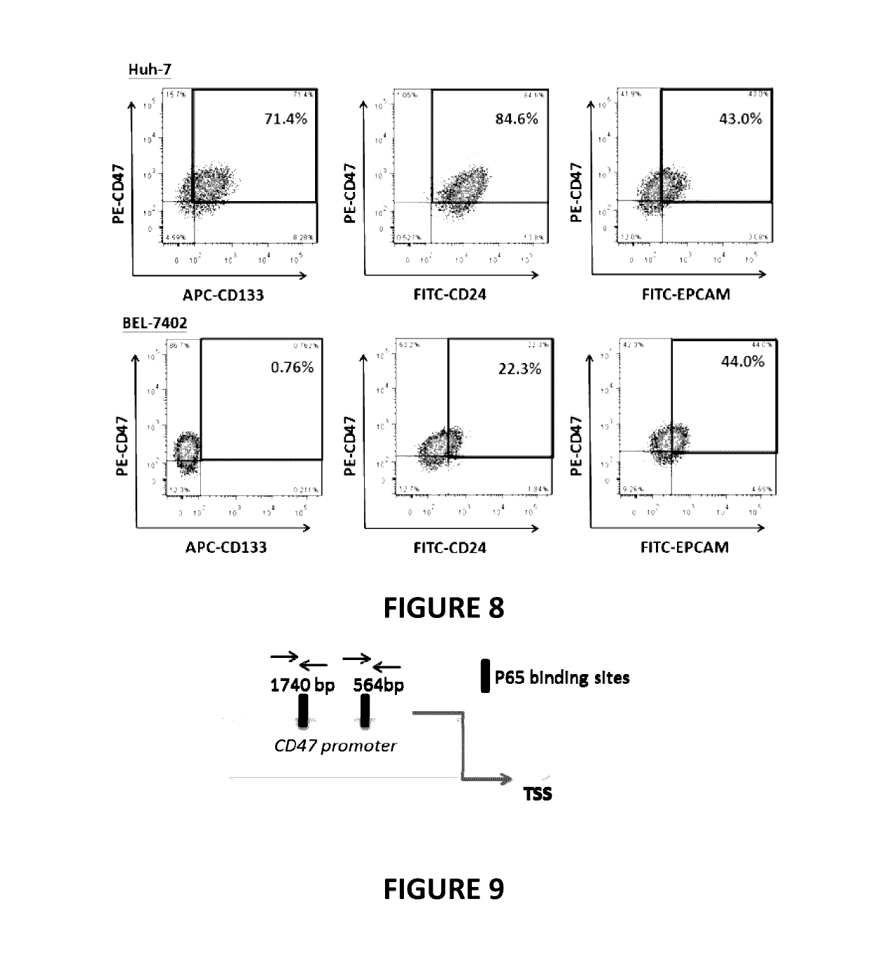

To verify whether sorafenib-resistant cells derived from Bel-7402 and Huh-7 cells had an enriched proportion of T-ICs, we first determined whether sorafenib-resistant HCC cells were more tumorigenic in vivo than their mock-control cells using a tumor-forming assay. A significant difference in tumor incidence was observed between these two groups of cells (FIG. 2(A)). As few as 100 and 500 sorafenib-resistant cells derived from Bel-7402 and Huh-7 cells, respectively, were sufficient for consistent tumor development in NOD/SCID mice (Table 1(A, B). To verify whether sorafenib at 100 mg/kg enriched the proportion of T-ICs in the residual PDTX#5 tumor, single HCC cells were isolated from untreated and sorafenib-resistant residual tumors. Tumor-forming abilities of HCC cells derived from these two groups were compared after serial subcutaneous (SC) transplantation of 1×10³ cells into NOD/SCID mice. Compared with HCC cells from untreated tumors, enhanced tumor-forming and self-renewal abilities were observed in sorafenib-resistant HCC cells from the residual tumor, which indicated successful enrichment of liver T-ICs in this sorafenib-resistant HCC PDTX#5 xenograft model (FIG. 2(B); Table 1(C)). To obtain further evidence for the self-renewal ability of sorafenib-resistant cells, we performed sphere formation assays. Compared to mock-control cells, significantly larger and more hepatospheres were observed in sorafenib-resistant cells isolated from Bel-7402, Huh-7, and PDTX#5 cells (FIG. 2(C)). Apart from these T-IC properties, we found that sorafenib-resistant cells displayed significantly higher migratory and invasive abilities in both Bel-7402 and Huh-7 cells (FIG. 2(D)). To identify a potential marker associated with sorafenib resistance for targeting T-ICs, we performed quantitative PCR (qPCR) analysis to compare the messenger RNA expression profiles of several known liver T-IC markers: CD24, CD47, CD133, and EpCAM. Interestingly, all four of the chosen markers were up-regulated in sorafenib-resistant clones. However, CD47 was found to be most significantly up-regulated in both Bel-7402 and Huh-7 resistant clones by 2.67- (P=0.01) and 17.30-fold (P=0.04), respectively (FIG. 2E)). Using flow cytometry analysis (FCA), we found that these CD47⁺ sorafenib-resistant HCC cells have a certain degree of overlap with other liver T-IC markers, including CD24, CD133, and EpCAM (FIG. 8). In addition, enrichment of the T-IC population was also evidenced by up-regulation of CD47 protein in sorafenib-resistant cells derived from Bel-7402, Huh-7, PDTX#1, and PDTX#5 cells (FIG. 2(F)).

TABLE 1
Tumorigenicity of sorafenib-resistant cells and mock-treated
HCC cells derived from Bel-7402, Huh-7 and PDTX#5 in
NOD/SCID mice. (A) Subcutaneous in vivo tumor development
in NOD/SCID mice of sorafenib-resistant cells and mock-treated
HCC from Bel-7402. (B) In vivo tumor development in NOD/SCID
mice of sorafenib-resistant cells and mock-treated HCC cells
from Huh-7 (C) Subcutaneous in vivo tumor development in
NOD/SCID mice of serially transplanted sorafenib-resistant
cells and mock-treated HCC cell-derived xenograft
tumors from PDTX#5.
A. Primary engraftment of Bel-7402 cells (3-4 weeks)

or/

| Cell | 200 | 1 | Total |
|---|---|---|---|
| Mock | 0/6 | 0 | 2/16 |
| Resista | 5/6 | 4 | 14/16 |

B. Primary engraftment of Huh-7 cells (8 weeks)

| | Tumor/ | | | |
|---|---|---|---|---|
| Cell | 50 | 1000 | 5 | Total |
| Mock | 1/6 | 1/6 | 0 | 2/18 |
| Resista | 4/6 | 6/6 | 4 | 14/18 |

C. Secondary engraftment of PDTX#5 cells (8 weeks)

Tumor/

| Cell no. | 5000 | 1000 | Total |
|---|---|---|---|
| Mock | 4/4 | 0/6 | 4/10 (40.0%) |
| Resista | 4/4 | 4/6 | 8/10 (80.0%) |

### Example 3

### Activation of NF-κB in Sorafenib-Resistant HCC Cells

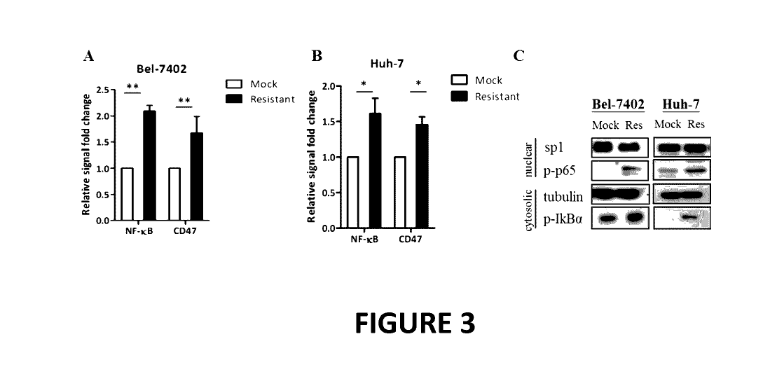

To examine whether CD47 was transcriptionally regulated, we examined CD47 promoter activity in sorafenib-resistant cells. Compared to mock-control cells, we found that sorafenib-resistant cells derived from Bel-7402 and Huh-7 cells exhibited increased CD47 promoter activity (FIG. 3(A),(B)). This result suggested that the increase in CD47 expression in sorafenib-resistant cells was a result of gene activation at the transcriptional level, rather than from protein stabilization. Based on bioinformatics analysis using the University of California Santa Cruz (UCSC) Genome Browser, we found two putative NF-κB-binding sites (2564 and 21,740 base pairs [bp]) upstream of the transcription start site of the CD47 promoter (FIG. 9). Therefore, we hypothesized that CD47 was transcriptionally activated by NF-κB. To test this hypothesis, we first examined whether NF-κB was activated in sorafenib-resistant cells using an NF-κB promoter assay. When compared to mock-control cells, we found that NF-κB activation was increased by 2.09- and 1.61-fold in sorafenib-resistant cells derived from Bel-7402 and Huh-7 cells, respectively (FIG. 3 (A),(B)). To further confirm NF-κB activation in sorafenib-resistant cells, we examined nuclear expression of p65 (Ser536) and cytosolic p-IκBα (Ser32/36) using western blotting analysis, given that activation of NF-κB is reflected by phosphorylation of both p65 and nuclear factor of kappa light polypeptide gene enhancer in B-cells inhibitor, alpha (IκB) by IκB kinase, which allows nuclear translocation of NF-κB from the cytoplasm (22). Consistent with enhanced NF-κB promoter activity, we found up-regulation of nuclear p65 (Ser536) and cytosolic p-IκBα (Ser32/36) in sorafenib-resistant Bel-7402 and Huh-7 cells (FIG. 3(C)).

### Example 3

### Alteration of NE-KB Levels Affects CD47 Expression

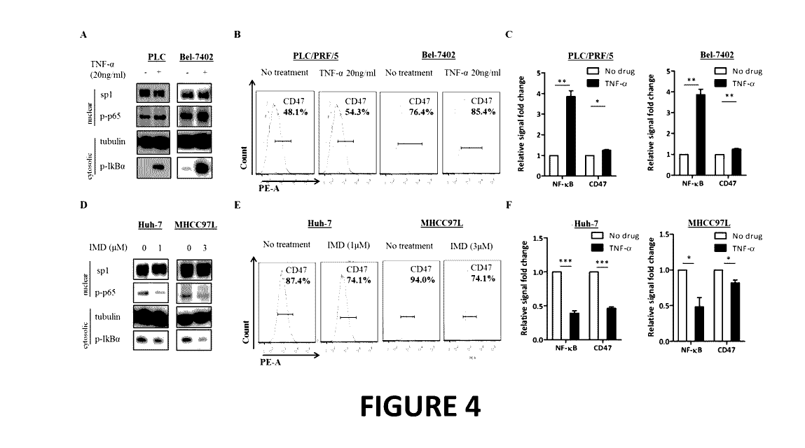

To examine the effect of altered NF-κB expression on CD47 expression, we first activated NF-κB in HCC cells which had lower CD47 expression (PLC/PRF/5 and Bel-7402) by treating with the NF-κB stimulator, tumor necrosis factor alpha (TNF-α). Upon treatment of HCC cells with TNF-α at a dose of 20 ng/mL, we successfully induced NF-κB activation, as evidenced by increased expression of nuclear p65 (Ser536) and cytosolic p-IκBα (Ser32/36); (FIG. 4(A)). Using FCA, we also detected an increase in CD47 expression on the surface of HCC cells (FIG. 4(B)). Consistently, an increase in NF-κB promoter activity was observed and this was accompanied by a corresponding increase in CD47 promoter activity upon treatment with TNF-α (FIG. 4(C)).

To further examine the regulatory role of NF-κB on CD47, we examined the effect of NF-κB inhibition in HCC cell lines with high CD47 expression (MHCC97L and Huh-7) by treatment with an NF-κB inhibitor (IMD-0354). Using western blotting analysis, we found that treatment with IMD-0354 at doses of 1 and 3 μM effectively suppressed NF-κB activation in Huh-7 and MHCC97L cells, respectively (FIG. 4(D)). Consistently, a reduction in cell-surface expression of CD47 was detected by FCA (FIG. 4(E)). Similarly, promoter activities of both NF-κB and CD47 were found to be significantly down-regulated upon treatment with IMD-0354 (FIG. 4(F)).

### Example 4

### Direct Regulatory Role of NE-jB on CD47 Expression

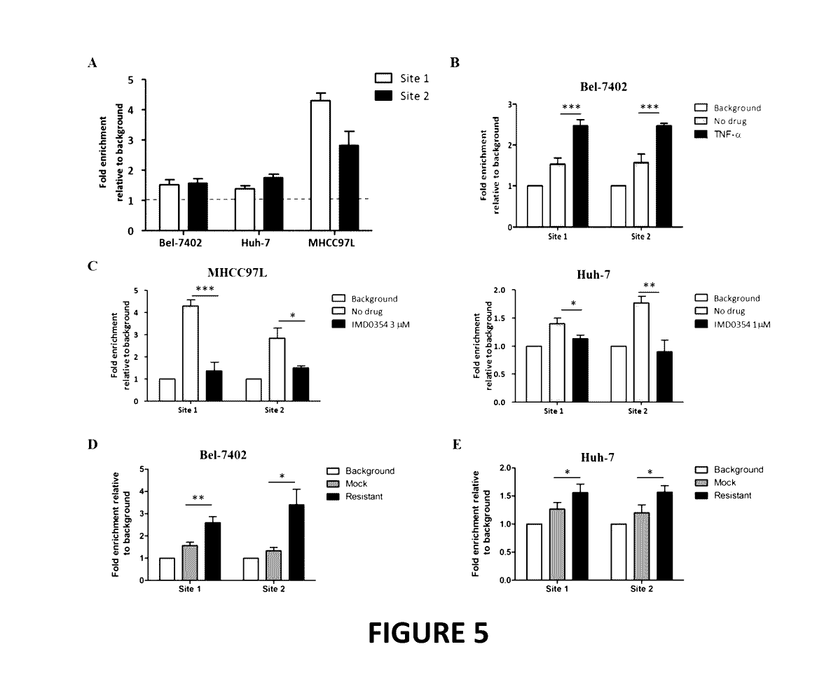

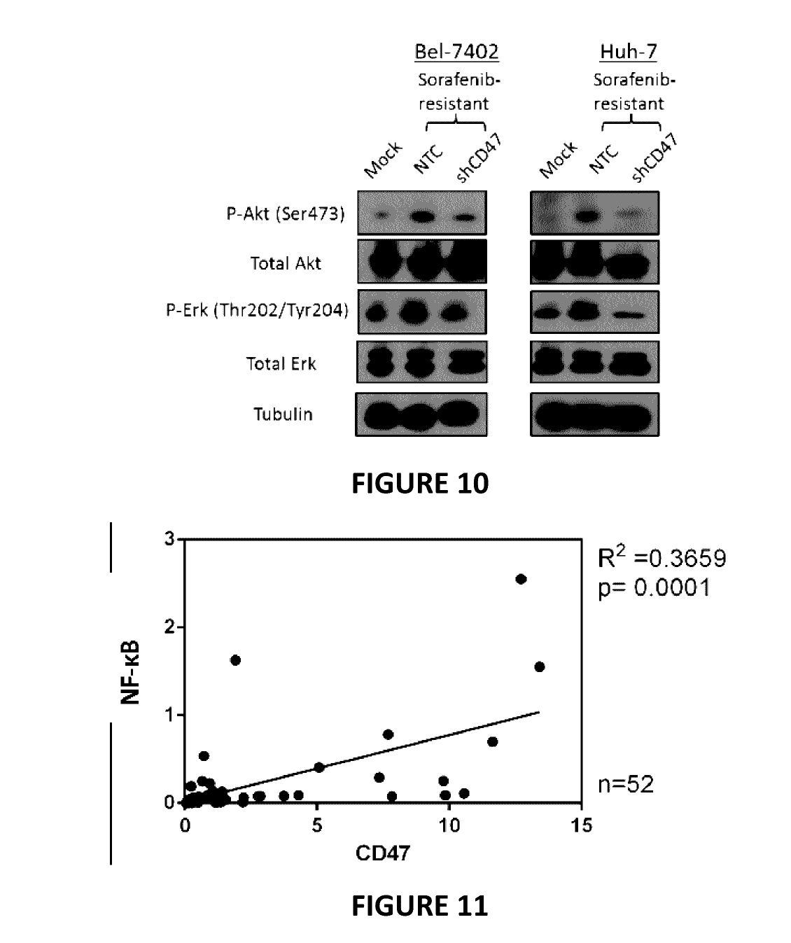

To determine the direct transcriptional regulatory role of NF-κB on CD47, we further examined potential binding between NF-jB and the CD47 promoter using a ChIP-qPCR assay. FIG. 5(A) demonstrates the binding between NF-κB and the CD47 promoter in Bel-7402, Huh-7, and MHCC97L cells, as evidenced by increased NF-κB occupancy when compared to the basal/background level and with the rabbit IgG control. To strengthen evidence in support of NF-κB playing a direct regulatory role on CD47 expression, IMD-0354 and TNF-α were applied to alter NF-κB expression to determine whether a change in site occupancy would occur. Addition of 20 ng/mL of TNF-α to Bel-7402 cells to up-regulate NF-κB, at 1 hour before fixation for ChIP, showed a significant increase in fold enrichment of NF-κB binding relative to background. At site 1 (2564 bp), this enrichment increased from 1.5- to 2.5-fold (P=0.004), whereas at site 2 (21,740 bp), enrichment increased from 1.6- to 2.5-fold (P=0.004; FIG. 5(B)). Conversely, Huh-7 and MHCC97L cells were treated with 1 and 3 μM of IMD-0354, respectively, for 24 hours before fixation for ChIP to inhibit NF-κB. Fold enrichment of binding was significantly decreased at both site 1 and 2 to near background level in both cell lines. In Huh-7 cells, fold enrichment at site 1 decreased from 1.4- to 1.1-fold (P=0.02), and at site 2, enrichment decreased from 1.8- to 0.9-fold (P=0.006). The effect of NF-κB inhibition was more evident in MHCC97L cells, where fold enrichment at site 1 decreased from 4.3- to 1.4-fold (P=0.0006), and at site 2, the decrease was from 2.8- to 1.5-fold (P=0.03; FIG. 5(C)). To further examine whether binding of NF-κB to CD47 promoter is increased in sorafenib-resistant HCC cells, we performed ChIP assay to compare site occupancy between sorafenib-resistant HCC cells and their mock counterparts. Interestingly, we found a significant increase in fold enrichment of NF-κB binding in sorafenib-resistant HCC cells. For Bel-7402, this enrichment increased from 1.5- to 2.6-fold (P=0.008) at site 1, whereas enrichment increased from 1.3- to 3.4-fold at site 2 (P=0.039; FIG. 5(D)). For Huh-7, this enrichment increased from 1.26- to 1.56-fold (P=0.024) at site 1, whereas enrichment increased from 1.2- to 1.57-fold at site 2 (P=0.013; FIG. 5(E)). These findings verified the direct binding of NF-κB to the CD47 promoter and provided evidence for the regulatory role of NF-κB. To further understanding the feedback mechanism of NF-κB-induced CD47 in regulation of liver T-ICs, we examined the upstream signaling molecules upstream of NF-κB before and after CD47 knockdown by western blotting analysis. Compared to mock-treated counterparts, we found that both phosphorylation of protein kinase B (Akt) and extracellular signal-regulated kinase were increased in sorafenib-resistant HCC cells, and this effect was abrogated when CD47 was repressed in these cells (FIG. 10). This result suggests the feedback mechanism of NF-κB-induced CD47 in regulation of liver T-ICs. To further confirm this observation in the clinical setting, we examined the correlation between NF-κB and CD47 levels in a cohort of 52 HCC patient samples using qPCR. Clinical information of these 52 HCC patient samples is provided in Table 2. Consistent with in vitro findings, we found that NF-κB expression significantly correlated with CD47 expression in these 52 HCC patient samples (P=0.0001; R²=0.3659, Pearson's correlation; FIG. 11. To evaluate the clinical significance of CD47 in HCC patients, clinicopathological analysis of these 52 HCC patients according to CD47 expression status was performed. Over-expression of CD47 expression was significantly correlated with absence of tumor encapsulation (P=0.014, Fisher's exact test) and presence of microsatellite formation (P=0.007, chi-square [χ²] test; Table 3.

TABLE 2
Clinical information of 52 HCC patients.

| Characteristics | Number of Patients |
|---|---|
| ^{#}Patients | 51 |
| Sex | |
| Male | 44 |
| Female | 7 |
| Age (Years) | Range = 24-74 |
| | (median = 51) |
| AFP (ng/ml) | Range = 2-492300 |
| | (median = 372) |
| HBV | |
| Negative | 5 |
| Positive | 41 |
| HCV | |
| Negative | 37 |
| Positive | 1 |
| Venous invasion | |
| Absent | 16 |
| Present | 34 |
| Tumor encapsulation | |
| Absent | 34 |
| Present | 15 |
| Tumor microsatellite formation | |
| Absent | 18 |
| Present | 31 |
| Direct liver invasion | |
| Absent | 29 |
| Present | 17 |
| Cellular differentiation | |
| Edmon 0-III | 20 |
| Edmon IV-VI | 29 |
| Tumor size (cm) | Range = 1.5-27 |
| ≦5 | 14 |
| >5 | 34 |
| Cirrhotic liver | |
| Normal and chronic hepatitis | 30 |
| Cirrhosis | 20 |
| pTNM stage | |
| I-II | 12 |
| III-IV | 35 |
| *Previous treatment history | |
| Yes | 2 |
| No | 47 |

^{#}Total 51 cases, 1 case information missing
*Previous treatment information.
1 case had right hepatic artery ligation
1 case had laparotomy
3 cases with no available information

TABLE 3
Clinicopathological correlation of CD47 in HCC patients.

CD47 expression

| Clinico-pathological variables | Low | High | P-value |
|---|---|---|---|
| ^{a}Gender | | | |
| Male | 22 | 22 | 1.000 |
| Female | 3 | 4 | |
| ^{b}Venous invasion | | | |
| Absent | 11 | 5 | 0.069 |
| Present | 14 | 20 | |
| ^{a}Tumor encapsulation | | | |
| Absent | 12 | 22 | 0.014* |
| Present | 11 | 4 | |
| ^{b}Tumor microsatellite formation | | | |
| Absent | 13 | 5 | 0.007* |
| Present | 10 | 21 | |
| ^{a}HBV | | | |
| Negative | 2 | 3 | 1.000 |
| Positive | 21 | 20 | |
| ^{a}HCV | | | |
| Negative | 21 | 16 | 0.447 |
| Positive | 0 | 1 | |
| ^{b}Direct liver invasion | | | |
| Absent | 16 | 13 | 0.193 |
| Present | 6 | 11 | |
| ^{b}Cellular differentiation | | | |
| Edmon 0-III | 11 | 9 | 0.348 |
| Edmon IV-VI | 12 | 17 | |
| ^{b}Tumor size | | | |
| ≦5 cm | 8 | 6 | 0.412 |
| >5 cm | 15 | 19 | |
| ^{b}Cirrhotic liver | | | |
| Normal and chronic hepatitis | 14 | 16 | 0.817 |
| Cirrhosis | 10 | 10 | |
| ^{a}pTNM stage | | | |
| I-II | 9 | 3 | 0.055 |
| III-IV | 15 | 20 | |

^{a}Fisher's Exact Test was used to calculate statistical significance
^{b}Pearson Chi-Square was used to calculate statistical significance
*Significant difference
Total 51 cases (1 case information missing)

### Example 5

### Suppression of NF-κB and CD47 Leads to Sorafenib Sensitization

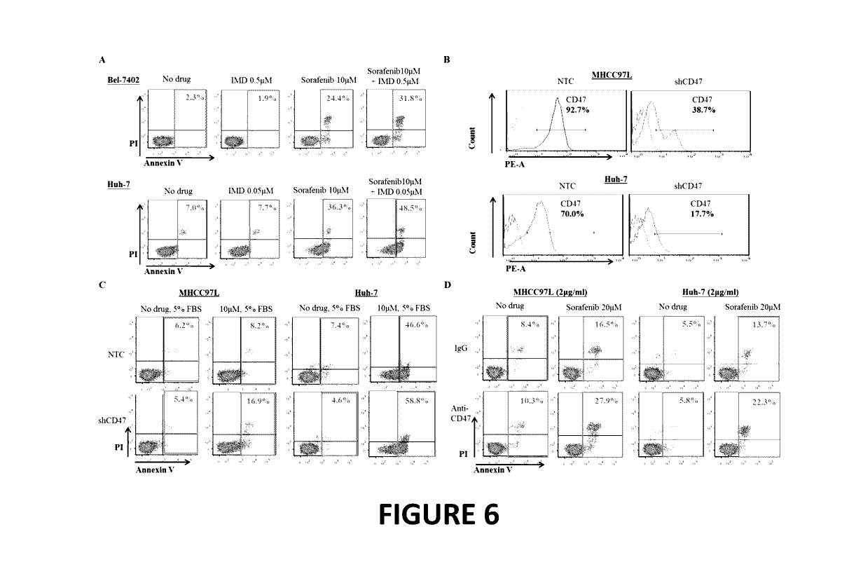

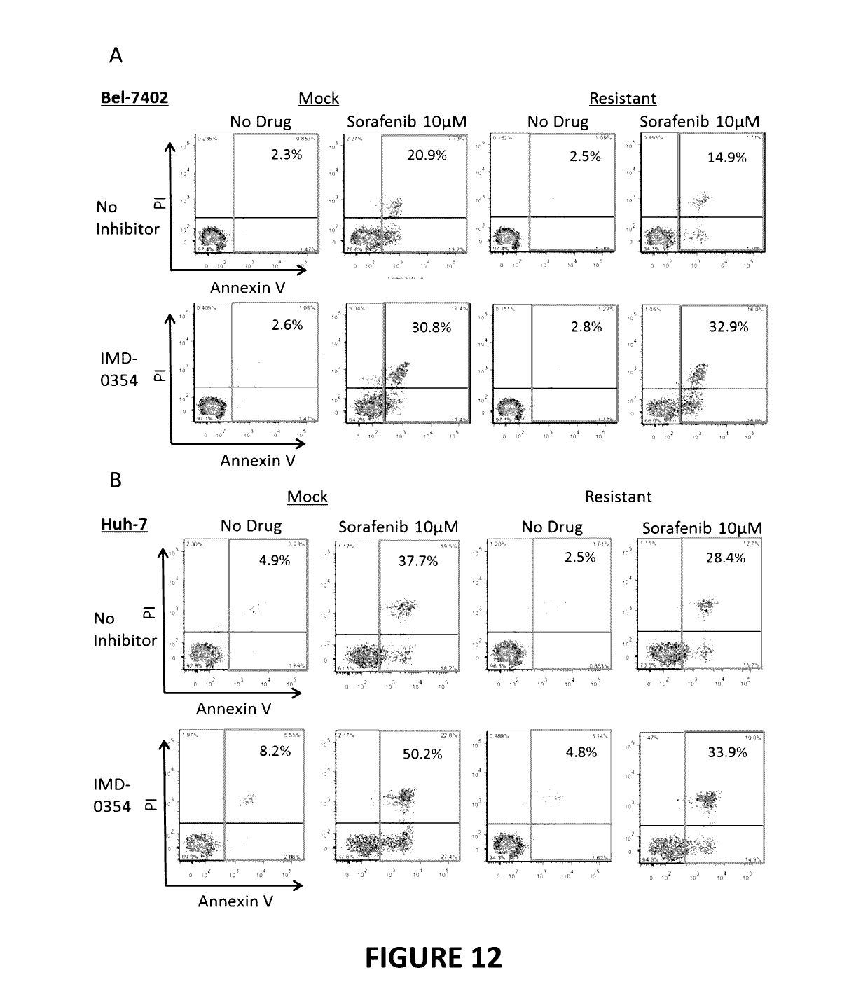

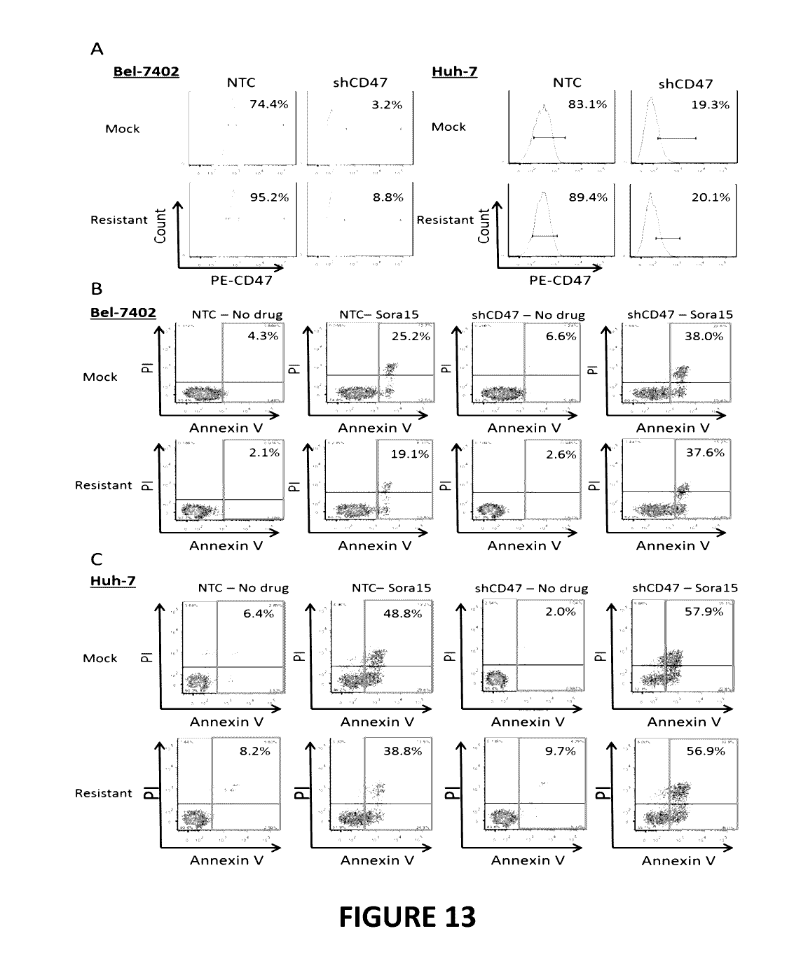

Based on the above findings, we hypothesized that NF-κB-mediated CD47 up-regulation promoted sorafenib resistance. To test this hypothesis, we first tested whether inhibition of NF-κB in HCC cells by IMD-0354 sensitized the effect of sorafenib. Treatment with 0.05 or 0.5 μM of IMD-0354, respectively, sensitized Huh-7 and Bel-7402 cells to sorafenib, but showed no toxic effects on cells (FIG. 6(A)). Interestingly, a similar sensitization effect was observed in mock-treated and sorafenib-resistant HCC cells (FIG. 12). Next, we examined the sensitization effect of CD47 suppression to sorafenib treatment. We performed a CD47 knockdown experiment using a lentivirus-based approach in cells highly expressing CD47 (Huh-7 and MHCC97L). After confirmation of successful CD47 knockdown in Huh-7 shCD47 and MHCC97L shCD47 clones by FCA (FIG. 6(B)), we examined the effect of CD47 suppression on sorafenib treatment at a dose of 10 μM. FIG. 6(C) shows that knockdown of CD47 in Huh-7 and MHCC97L cells increased the sensitivity of cells to sorafenib. To further examine whether a similar effect would be observed in sorafenib-resistant cells derived from Bel-7402 and Huh-7, we repressed CD47 expression in these cells and performed an apoptotic assay upon sorafenib treatment. After successful knockdown of CD47 expression, CD47 expression was found to be comparable between mock-treated and sorefenib-resistant HCC cells. Consistently, we found that CD47-repressed cells showed increased sensitivity to sorafenb, when compared to nontarget control cells (FIG. 13). These results showed that CD47 expression level is crucial to determine sorafenib sensitivity in HCC cells.

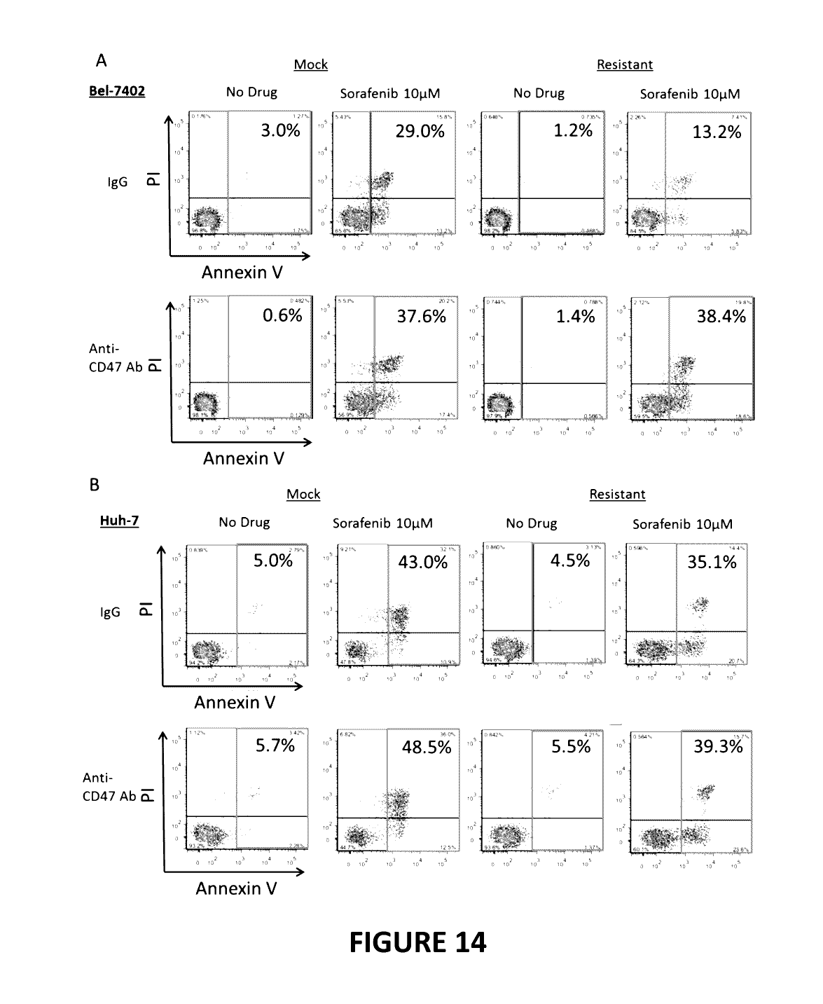

Recently, anti-CD47 Ab was found to be a novel therapeutic target for human solid tumors (20). Therefore, we examined the combined in vitro effect of purified anti-CD47 Ab (clone B6H12) and sorafenib in cells highly expressing CD47. Interestingly, we found that anti-CD47 Ab at a dose of 2 mg/mL sensitized Huh-7 and MHCC97L cells to sorafenib (FIG. 6D). In addition, we also examined whether anti-CD47 Ab also had an effect on sorafenib-resistant HCC cells. At a dose of 2 mg/mL, we found a similar sensitization effect between mock-treated and sorafenib-resistant HCC cells, and the effect was more prominent in sorafenib-resistant cells derived from Bel-7402 cells (FIG. 14). This in vitro result suggests that the sensitization effect of the anti-CD47 Ab is independent of CD47/signal regulatory protein alpha interaction.

### Example 6

### Combination of Anti-CD47 Ab and Sorafenib Results in Sorafenib Sensitization in a PDTX Model

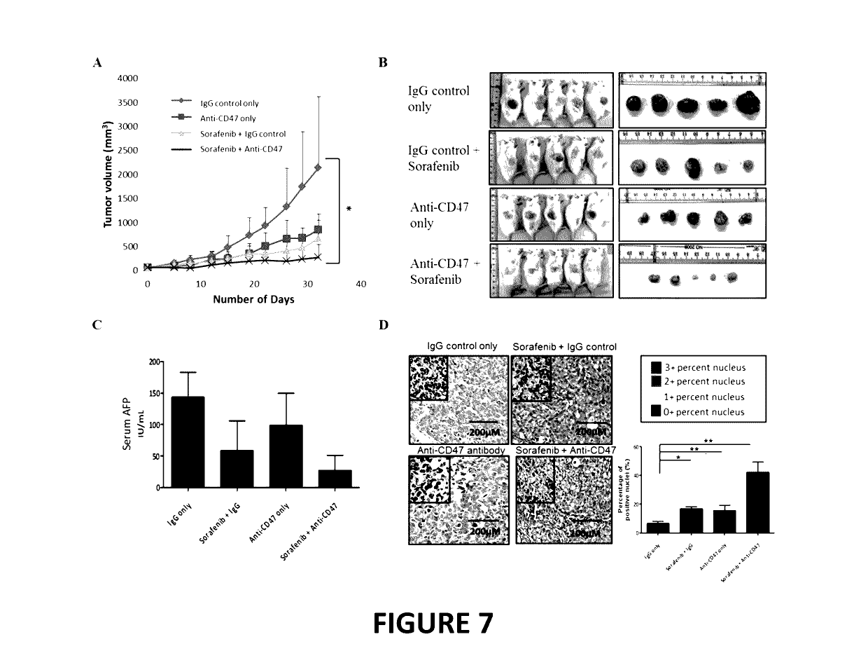

Given the crucial role of CD47 in regulating sorafenib resistance, we examined the therapeutic role of targeting CD47 alone and its combined effect with sorafenib in vivo using the PDTX#5 and anti-CD47 mAb. The following therapeutic regimens were used: (1) 500 μg/mouse anti-CD47 Ab; (2) 100 mg/kg of sorafenib plus IgG control; (3) 100 mg/kg of sorafenib plus 500 mg/mouse anti-CD47 Ab; or (4) IgG control. Treatment was begun once the size of the xenografts had reached approximately 5×5 mm. During the experiment, no signs of toxicity (infection, diarrhea, or loss of body weight) were observed in animals undergoing anti-CD47 Ab treatment. Tumors and their corresponding volumes are shown in FIG. 7(A),(B). Anti-CD47 Ab suppressed tumor volumes in a manner similar to the effect of sorafenib. In addition, combined anti-CD47 Ab and sorafenib exerted a synergistic effect, resulting in maximal suppression of tumors, when compared to the control group. Alpha-fetoprotein (AFP) has recently been considered a liver T-IC marker and has commonly been used as a cancer marker for diagnosis and prognosis of HCC (23). Therefore, we extracted serum from blood of mice in the different treatment groups and observed the effect of anti-CD47 Ab and its combined effect with sorafenib on secretory level of AFP. Consistent with tumor volume, we found that the greatest reduction of serum AFP level in mice occurred when they were cotreated with anti-CD47 Ab and sorafenib (FIG. 7(C)). To further examine whether sorafenib sensitization was a result of increased apoptosis, we performed terminal deoxynucleotidyl transferase dUTP nick end labeling (TUNEL) assays to assess the percentage of apoptotic nuclei in tumors of the different treatment groups. Percentage of apoptotic nuclei in tumor tissues treated with anti-CD47 Ab or sorafenib was higher, when compared to untreated samples (FIG. 7(D)). Furthermore, combined anti-CD47 Ab and sorafenib treatment showed a remarkable difference in inducing tumor cell apoptosis, when compared with sorafenib alone (FIG. 7(D)).

### Discussion

Establishment of different sorafenib-resistant HCC cell lines has previously been successful in vitro, and these cells were found to undergo Akt and mammalian target of rapamycin activation (24). Phenotypically, sorafenib-resistant HCC cells exhibited enhanced metastatic potential upon long-term exposure to sorafenib (25). However, thus far, no reports have been published on in vivo establishment of sorafenib-resistant HCC cells using patient-derived xenografts in mice. This invention shows that sorafenib-resistant cells derived from Huh-7 and Bel-7402 cells exhibited T-IC properties, including enhanced invasiveness, self-renewal capacity, and tumorigenicity, when compared to their mock counterparts. Consistent with this finding, a similar observation was observed in sorafenib-resistant cells derived from two patient-derived xenografts. This result is in line with the finding that label-retaining liver cancer cells were resistant to sorafenib (26). Recently, we have identified CD47 as an attractive therapeutic target for liver T-ICs, given that it is preferentially expressed in liver T-ICs (17). Therefore, we also examined CD47 expression in sorafenib-resistant HCC cells. Interestingly, CD47 expression was found to be consistently up-regulated in all four established sorafenib-resistant HCC cell lines, showing the therapeutic benefit of targeting CD47 together with sorafenib treatment.

Thus far, the regulatory mechanism for CD47 expression remains unknown. The only reported regulator of CD47 expression is nuclear respiratory factor 1, a transcription factor (27). To determine whether CD47 was transcriptionally regulated, we first examined CD47 promoter activity in sorafenib-resistant cells. Compared to mock-control cells, we found that CD47 promoter activity was increased in sorafenib-resistant HCC cells. Based on bioinformatics analysis using the UCSC Genome Browser, we found two putative NF-κB-binding sites upstream of the transcription start site of the CD47 promoter. Using ChIP assays, we demonstrated, for the first time, that NF-κB directly binds to the promoter of CD47. Application of IMD-0354 and TNF-α was then used to examine their effects on NF-κB binding efficiency, which demonstrated that, upon stimulation of NF-κB, both sites showed increased fold enrichment in site occupation. Conversely, inhibition of NF-κB showed decreased fold enrichment of site occupation. These findings confirmed the direct binding of NF-κB to the CD47 promoter. In addition, in a cohort of HCC clinical samples, NF-κB expression was consistently correlated with that of CD47.

NF-κB has long been connected to inflammation leading to HCC, and its activation is an early event that is frequently observed in hepatocarcinogenesis (28). Moreover, NF-κB activity was recently reported to increase upon sorafenib treatment (29). Using luciferase reporter assays, we found that NF-κB promoter activity was enhanced in sorafenib-resistant clones, suggesting that the NF-κB-signaling pathway was involved in development of resistance to sorafenib. Further confirmation by western blotting analysis also showed NF-κB activation, as evidenced by increased phosphorylation of p65 (Ser 536), in the nucleus and of IκBα (Ser 32/36) in cytoplasm. NF-κB was found to play a crucial role in maintenance of T-ICs in leukemia and breast cancer (29,30). In addition, combined treatment with the NF-κB inhibitor, sulforaphane, and sorafenib had a synergistic effect in elimination of pancreatic T-ICs (31). However, few studies have been performed on liver T-ICs. By co-treating HCC cells with the NF-κB inhibitor, IMD-0354, and sorafenib, we showed that suppression of NF-κB sensitized HCC cells to sorafenib treatment.

To examine the role of CD47 in sorafenib resistance, we stably suppressed CD47 expression in two cell lines highly expressing CD47 using a lentivirus-based approach. The results from the apoptosis assay revealed that MHCC97L and Huh-7 cells showed a greater number of apoptotic cells undergoing apoptosis after CD47 knockdown and treatment with 10 μM of sorafenib, suggesting that CD47 plays a functional role in sorafenib sensitivity in HCC. Likewise, ligation of CD47 to an anti-CD47 Ab also exerted a similar effect in the same two cell lines. Given that macrophages were not found in these in vitro experiments, this invention suggests that a macrophage-independent mechanism, which involves CD47 signaling, might be involved in this sensitization to sorafenib. Aside from its antiphagocytic functions, we previously reported that anti-CD47 Ab suppressed CTSS/PAR2 signaling, (17) which suggests that the sensitization effect of CD47 Ab to sorafenib may be a result of inhibition of CTSSPAR2 signaling. In addition, CD47 was previously reported by other groups to bind to thrombospondin 1, (32) which is a known protease. Therefore, CD47 may be able to affect many yet unknown pathways that lead to sorafenib sensitization.

No reports have shown the combined effect of an anti-CD47 Ab with other chemotherapeutic or molecule-targeted drugs. By using the PDTX HCC model, results from this experiment were very encouraging, given that sorafenib and anti-CD47 Ab treatment on their own were able to produce only a 2.5- to 3.2-fold decrease in tumor volume, whereas combined treatment produced a remarkable 7.8-fold suppression. This observation shows a synergistic effect of the combination treatment of sorafenib and anti-CD47 Ab. The therapeutic efficacy of sorafenib was also evidently improved upon combination therapy. Moreover, no visible indications of toxic effects resulting from anti-CD47Ab were observed in mice over the 31-day treatment period. The IgG group was found to have the fewest number of apoptotic nuclei, whereas the cotreatment group was found to have the greatest number of apoptotic nuclei, which confirmed that apoptosis was involved in sorafenib sensitization in vivo. Serum AFP levels in the four groups of mice also positively correlated with inhibition of tumor growth.

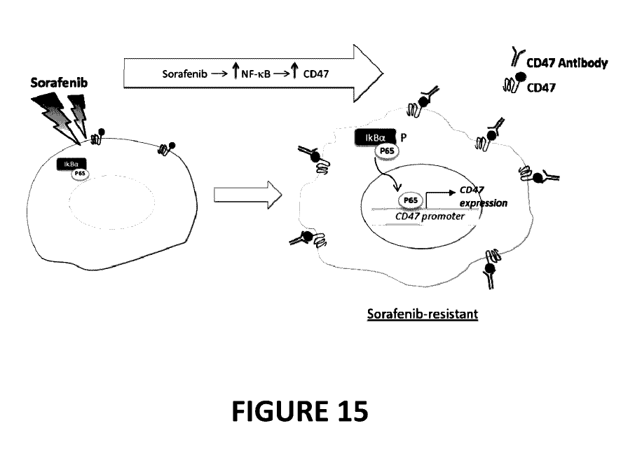

In conclusion, the present invention shows that sorafenib resistance is driven by enrichment of CD471 liver T-ICs, which is mediated through NF-κB activation (FIG. 15). Further, administration of an anti-CD47 Ab suppressed in vivo tumor growth and exerted a synergistic effect with sorafenib treatment. Thus, the methods of the present invention which target T-ICs expressing CD47 with an anti-CD47 Ab in combination with sorafenib provide a novel therapeutic strategy for improving efficacy of sorafenib.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Siegel R, Naishadham D, Jemal A. Cancer statistics 2013. CA Cancer J Clin 2013; 63:11-30.
2. Llovet J M, Di Bisceglie A M, Bruix J, Kramer B S, Lencioni R, Zhu A X, et al. Panel of Experts in HCC-Design Clinical Trials. Design and endpoints of clinical trials in hepatocellular carcinoma. J Natl Cancer Inst 2008; 100:698-711.
3. Llovet J M, Ricci S, Mazzaferro V, Hilgard P, Gane E, Blanc J F, et al. Sorafenib in advanced hepatocellular carcinoma. N Engl J Med 2008; 359:378-390.
4. Cheng A L, Kang Y K, Chen Z. Tsao C J, Qin S, Kim J S, et al. Efficacy and safety of sorafenib in patients in the Asia-Pacific region with advanced hepatocellular carcinoma: a phase III randomised, double-blind, placebo-controlled trial. Lancet Oncol 2009; 10:25-34.
5. Yau T, Yao T J, Chan P, Wong H, Pang R, Fan S T, et al. The significance of early alpha-fetoprotein level changes in predicting clinical and survival benefits in advanced hepatocellular carcinoma patients receiving sorafenib. Oncologist 2011; 16:1270-1279.
6. Villanueva A, Llovet J M. Second-line therapies in hepatocellular carcinoma: emergence of resistance to sorafenib. Clin Cancer Res 2012; 18: 1824-1826.
7. Shan J, Shen J, Liu L, Xia F, Xu C, Duan G, et al. Nanog regulates self-renewal of cancer stem cells through the insulin-like growth factor pathway in human hepatocellular carcinoma. HEPATOLOGY 2012; 56:1004-1014.
8. Chen X, Lingala S, Khoobyari S, Nolta J, Zern M A, Wu J. Epithelial mesenchymal transition and hedgehog signaling activation are associated with chemoresistance and invasion of hepatoma subpopulations. J Hepatol 2011; 55:838-845.
9. Ma S, Chan K W, Hu L, Lee T K, Wo J Y, Ng I O, et al. Identification and characterization of tumorigenic liver cancer stem/progenitor cells. Gastroenterology 2007; 132:2542-2556.
10. Yamashita T, Budhu A, Forgues M, Wang X W. Activation of hepatic stem cell marker EpCAM by Wnt-beta-catenin signaling in HCC. Cancer Res 2007; 67:10831-20839.
11. Yang Z F, Ho D W, Ng M N, Lau C K, Yu W C, Ngai P, et al. Significance of CD901 cancer stem cells in human liver cancer. Cancer Cell 2008; 13:153-166.
12. Haraguchi N, Ishii H, Mimori K, Tanaka F, Ohkuma M, Kim H M, et al. CD13 is a therapeutic target in human liver cancer stem cells. J Clin Invest 2010; 120:3326-3339.
13. Ma S, Lee T K, Zheng B J, Chan K W, Guan X Y. CD1331 HCC cancer stem cells confer chemoresistance by preferential expression of the Akt/PKB survival pathway. Oncogene 2008; 27:1749-1758.
14. Lee T K, Castilho A, Cheung V C, Tang K H, Ma S, Ng I O. CD241 liver tumor-initiating cells drive self-renewal and tumor initiation through Stat3-mediated nanog regulation. Cell Stem Cell 2011; 9:50-63.
15. Wilson G S, Hu Z, Duan W, Tian A, Wang X M, McLeod D, et al. LCSCs Efficacy of using cancer stem cell markers in isolating and characterizing liver cancer stem cells. Stem Cells Dev 2013; 22: 2655-2664.
16. Xin H W, Ambe C M, Hari D M, Wiegand G W, Miller T C, Chen J Q, et al. Label-retaining liver cancer cells are relatively resistant to sorafenib. Gut 2013; 62:1777-1786.
17. Lee T K, Cheung V C, Lu P, Lau E Y, Ma S, Tang K H, et al. Blockade of CD47 mediated CTSS-PAR2 signaling provides a therapeutic target for hepatocellular carcinoma. HEPATOLOGY 2014; 60:179-191.
18. Majeti R, Chao M P, Alizadeh A A, Pang W W, Jaiswal S, Gibbs K D, Jr., et al. CD47 is an adverse prognostic factor and therapeutic antibody target on human acute myeloid leukemia stem cells. Cell 2009; 138:286-299.
19. Chan K S, Espinosa I, Chao M, Wong D, Ailles L, Diehn M, et al. Identification, molecular characterization, clinicalprognosis, and therapeutic targeting of human bladder tumor-initiating cells. Proc Natl Acad Sci USA 2009; 106:14016-14021.
20. Willingham S B, Volkmer J P, Gentles A J, Sahoo D, Dalerba P, Mitra S S, et al. The CD47-signal regulatory protein alpha (SIPRa) interaction is a therapeutic target for human solid tumors. Proc Natl Acad Sci USA 2012; 109:6662-6667.
21. Tseng D, Volkmer J P, Willingham S B, Contreras-Trujillo H, Fathman J W, Fernhoff N B, et al. Anti-CD47 antibody-mediated phagocytosis of cancer by macrophage primes an effective antitumor T-cell response. Proc Natl Acad Sci USA 2013; 110:11103-11108.
22. Schmitz M L, Mattioli I, Buss H, Kracht M. NF-kappaB: a multifac-eted transcription factor regulated at several levels. Chembiochem 2004; 5:1348-1358.
23. Yamashita T, Forgues M, Wang W, Kim J W, Ye Q, Jia H, et al. EpCAM and alpha-fetoprotein expression defines novel prognostic subtypes of hepatocellular carcinoma. Cancer Res 2008; 68:1451-1461.
24. Zhai B, Hu F, Jiang X, Xu J, Zhao D, Liu B, et al. Inhibition of akt reverses the acquired resistance to sorafenib by switching protective autophagy to autophagic cell death in hepatocellular carcinoma. Mol Cancer Ther 2014; 13:1589-1598.
25. Chow A K, Ng L, Lam C S, Wong S K, Wan T M, Cheng N S, et al. The Enhanced metastatic potential of hepatocellular carcinoma (HCC) cells with sorafenib resistance. PLoS One 2013; 8:e78675.
26. Xin H W, Ambe C M, Hari D M, Wiegand G W, Miller T C, Chen J Q, et al. Label-retaining liver cancer cells are relatively resistant to sorafenib. Gut 2013; 62:1777-1786.
27. Chang W T, Huang A M. Alpha-Pal/NRF-1 regulates the promoter of the human integrin-associated protein/CD47 gene. J Biol Chem 2004; 279:14542-14550.
28. Liu P, Kimmoun E, Legrand A, Sauvanet A, Degott C, Lardeux B, et al. Activation of NF-kappa B, AP-1 and STAT transcription factors is a frequent and early event in human hepatocellular carcinomas. J Hepatol 2002; 37:63-71.
29. Kuo H P, Wang Z, Lee D F, Iwasaki M, Duque-Afonso J, Wong S H, et al. Epigenetic roles of MLL oncoproteins are dependent on NF-jB. Cancer Cell 2013; 24:423-437.
30. Yamamoto M, Taguchi Y, Ito-Kureha T, Semba K, Yamaguchi N, Inoue J. NF-jB non-cell-autonomously regulates cancer stem cell populations in the basal-like breast cancer subtype. Nat Commun 2013; 4:2299.
31. Rausch V, Liu L, Kallifatidis G, Baumann B, Mattern J, Gladkich J, et al. Synergistic activity of sorafenib and sulforaphane abolishes pancreatic cancer stem cell characteristics. Cancer Res 2010; 70:5004-5013.
32. Maxhimer J B, Soto-Pantoja D R, Ridnour L A, Shih H B, Degraff W G, Tsokos M, et al. Radioprotection in normal tissue and delayed tumor growth by blockade of CD47 signaling. Sci Transl Med 2009; 1:3ra7.

We claim:

1. A method for treating or inhibiting liver cancer, comprising administering to a subject in need thereof an effective amount of:
sorafenib; and
an inhibiting agent selected from a CD47 inhibiting agent, a NF-κB inhibiting agent, and combinations thereof.

2. The method of claim 1, wherein the CD47 inhibiting agent is an anti-CD47 antibody or antibody fragment thereof.

3. The method of claim 2, wherein the anti-CD47 antibody is a chimeric, human, or humanized antibody.

4. The method of claim 2, wherein the antibody fragment is selected from Fab, Fab′, Fab′-SH, F (ab′) 2, Fv, diabodies, single-chain antibody fragment, and a multispecific antibody comprising multiple different antibody fragments.

5. The method of claim 2, wherein the anti-CD47 antibody is conjugated or covalently bound to a toxic moiety.

6. The method of claim 2, wherein the anti-CD47 antibody is conjugated or covalently bound to a detectable moiety.

7. The method of claim 6, wherein the detectable moiety is a radiolabel.

8. The method of claim 2, wherein the anti-CD47 antibody is a monoclonal antibody.

9. The method of claim 1, wherein the NF-κB inhibiting agent is IMD-0354.

10. The method of claim 1, wherein the subject is a human.

11. The method of claim 1, wherein the liver cancer is hepatocellular carcinoma.

12. A method of preventing recurrence of or treating liver cancer in a subject, the method comprising:
administering to the subject an effective amount of sorafenib; and
an inhibiting agent selected from a CD47 inhibiting agent, a NF-κB inhibiting agent, and combinations thereof,
wherein the subject had surgical resection of a primary liver tumor, had undergone hepatectomy, and/or had liver transplantation.

13. The method of claim 12, wherein the CD47 inhibiting agent is an anti-CD47 antibody or antibody fragment thereof.

14. The method of claim 12, wherein the NF-κB inhibiting agent is IMD-0354.

15. A method for increasing sensitivity of liver cancer cells to sorafenib treatment, comprising contacting the cells with an inhibiting agent selected from a CD47 inhibiting agent, a NF-κB inhibiting agent, and combinations thereof, either contemporaneously with, prior to, or after sorafenib treatment.

16. The method of claim 15, wherein the liver cancer cells comprise tumor-initiating cells.

17. The method of claim 15, wherein the CD47 inhibiting agent is an anti-CD47 antibody or antibody fragment thereof.

18. The method of claim 15, wherein the NF-κB inhibiting agent is IMD-0354.

19. A pharmaceutical composition comprising sorafenib; an inhibiting agent selected from a CD47 inhibiting agent, a NF-κB inhibiting agent, and combinations thereof; and a pharmaceutically acceptable carrier.

20. The pharmaceutical composition of claim 19, wherein the CD47 and NF-κB inhibiting agents are selected from a respective antibody, a respective binding partner, a respective binding aptamer, an antisense nucleic acid molecule that inhibits the expression of the respective protein, a compound that inhibits the respective protein, and combinations thereof.


SEQUENCE LISTING (16 sequences)

SEQ ID NO: 1 - DNA, 20 bp, Homo sapiens
gtggggcgccccaggcacca

SEQ ID NO: 2 - DNA, 24 bp, Homo sapiens
ctccttaatgtcacgcacgatttc

SEQ ID NO: 3 - DNA, 24 bp, Homo sapiens
tgaagaacatgtgagaggtttgac

SEQ ID NO: 4 - DNA, 24 bp, Homo sapiens
gaaaactgaatctccattccacaa

SEQ ID NO: 5 - DNA, 23 bp, Homo sapiens
caatcacgtaagggtctcatagg

SEQ ID NO: 6 - DNA, 20 bp, Homo sapiens
gatggactccgatttggaga

SEQ ID NO: 7 - DNA, 22 bp, Homo sapiens
tggatgcagaacttgacaacgt

SEQ ID NO: 8 - DNA, 23 bp, Homo sapiens
atacctgctacgacagtcgtggt

SEQ ID NO: 9 - DNA, 21 bp, Homo sapiens
tgatcctgactgcgatgagag

SEQ ID NO: 10 - DNA, 21 bp, Homo sapiens
cttgtctgttcttctgacccc

SEQ ID NO: 11 - DNA, 18 bp, Homo sapiens
agcaccatcaactatgat

SEQ ID NO: 12 - DNA, 18 bp, Homo sapiens
gagttatagcctcagggt

SEQ ID NO: 13 - DNA, 20 bp, Homo sapiens
ggacacctaggctttcacca

SEQ ID NO: 14 - DNA, 20 bp, Homo sapiens
agggaagagcagagcgagta

SEQ ID NO: 15 - DNA, 20 bp, Homo sapiens
agagcagctggttggctaaa

SEQ ID NO: 16 - DNA, 20 bp, Homo sapiens
gaacatcacccgatctccac